SEQUENCE LISTING (4 sequences)

SEQ ID NO: 1 - DNA, 1578 bp, Candida albicans
  CDS (1)..(1578)
  misc_feature (1473)..(1473): n is a, c, g, or t
atgtcaacgtattctataccctattacaatcaaatgaacggaaattacaataacggtatgccccagcaaacaactgcagccaatcaacaggcttttcctcagcaacaacaaccaacaacaacaggcaatgctagccaacagcaacagcaagcagctgctacggcagctgcagctgcagtccaacagccttataactacatgttctatcaacaacaaggacaaccaggtcaacagactggacagacagcaggacaacaacaacaacagcagcagcagcagcaacaatatgattacaatacctacaacagatatcagtatcctgccgcaacatctcaaggaaactattaccagcaaacaattcctaatcaattgtcacagccacaacctcagcattacaatggatctaatcgtaattacacaagtgctcctagtggtgcccccataccttccaattctaccagtggaccttcacaacagccaccactaccaggtcaacaagcagtacctatcccaccacatgtatcgacaatgcaacaaccaactcctgttcaggatacgttgaacgcctcgagcacttccactgtggggcaattccaaccaccaggaatcagaccacgagtaacaactaccatgtgggaagatgaaaaaactttgtgctatcaagttgatgccaataatgtgtcggttgtcagaagagcagataataatatgattaacggaaccaaattgctcaatgtggcccaaatgacacgtggtagaagagatgggattttgaaatcagaaaaggtgagacacgttgtgaaaatcggatcaatgcatttgaaaggagtctggattccatttgaaagagcattggccatggctcaacgtgaacaaattgtggatatgttgtatcctttgtttgtcagagatattaaacgagtgattcaaaccggagtaactcctaatgcagctgctgcaacggccgccgccgctgccactgccacttctgcttcggctcctccacctccacctccacccgttgctgctgctactactactgctgctactgctatttccaaaagttctagcggtaatgggaacagtatatctgctaccagtggtggcagtaatgtgtctggtgcttctggtgcaggttccaccactagtccggtaaataccaaggctgccactgctgctggtatccctcaaggtaattattatcaaacttacaaccaacagcagtatcctcaacagtatggtcagtataatgctcctggtaagaaccaaaatacacctgcatcacaaccaggttctacaaccaatgatcaatatttacaacaacaacagcaacaacaacaacaaatgtatgggtatcacgtgaatatttccagggtggtgctgctaatagtagttctagcccaattattatcacgacagcaccaatgtatgcatcatcatatccataccacagcacgacaaaagcaacagcancacgaccaaatcacaacgaacagtcagatcagcancaagcttctacaccaagtggtggtgcagaaactagatctgtgcaccaatcaccccaagttcagtcattga

SEQ ID NO: 2 - protein, 525 aa, Candida albicans
  misc_feature (491)..(491): The ′Xaa′ at location 491 stands for Gln, or His.
MSTYSIPYYNQMNGNYNNGMPQQTTAANQQAFPQQQQPTTTGNASQQQQQAAATAAAAAVQQPYNYMFYQQQGQPGQQTGQTAGQQQQQQQQQQQYDYNTYNRYQYPAATSQGNYYQQTIPNQLSQPQPQHYNGSNRNYTSAPSGAPIPSNSTSGPSQQPPLPGQQAVPIPPHVSTMQQPTPVQDTLNASSTSTVGQFQPPGIRPRVTTTMWEDEKTLCYQVDANNVSVVRRADNNMINGTKLLNVAQMTRGRRDGILKSEKVRHVVKIGSMHLKGVWIPFERALAMAQREQIVDMLYPLFVRDIKRVIQTGVTPNAAAATAAAAATATSASAPPPPPPPVAAATTTAATAISKSSSGNGNSISATSGGSNVSGASGAGSTTSPVNTKAATAAGIPQGNYYQTYNQQQYPQQYGQYNAPGKNQNTPASQPGSTTNDQYLQQQQQQQQQMYGYHVNISRVVLLIVVLAQLLSRQHQCMHHHIHTTARQKQQXHDQITTNSQISXKLLHQVVVQKLDLCTNHPKFSH

SEQ ID NO: 3 - DNA, 20 bp, Artificial sequence
  Forward primer Efg1
ctgcttcggctcctccacct

SEQ ID NO: 4 - DNA, 22 bp, Artificial Sequence
  Reverse primer Efg1
cctgcaccagaagcaccagaca

(19) United States
(12) Patent Application Publication
(10) Pub. No.: US 2014/0079741 A1
(43) Pub. Date: Mar. 20, 2014

(54) INHIBITION AND TREATMENT OF BIOFILMS

(71) Applicants: Anna Bink, Westerlo (BE); Bruno Cammue, Alsemberg (BE); Johan Martens, Huldenberg (BE); Bram Neirinck, Izegem (BE); Karin Thevissen, Bierbeek (BE); Jozef Vleugels, Olen (BE)

(72) Inventors: Anna Bink, Westerlo (BE); Bruno Cammue, Alsemberg (BE); Johan Martens, Huldenberg (BE); Bram Neirinck, Izegem (BE); Karin Thevissen, Bierbeek (BE); Jozef Vleugels, Olen (BE)

(73) Assignee: KATHOLIEKE UNIVERSITEIT LEUVEN KU LEUVEN RESEARCH & DEVELOPMENT, Leuven (BE)

(21) Appl. No.: 14/006,074

(22) PCT Filed: Mar. 19, 2012
(86) PCT No.: PCT/EP12/54843

§ 371 Date: Dec. 2, 2013

Related U.S. Application Data
(60) Provisional application No. 61/517,212, filed on Apr. 15, 2011.

(30) Foreign Application Priority Data
Mar. 18, 2011 (GB) 1104584.6
Dec. 22, 2011 (GB) 1122124.9

(51) Int. Cl. A61L 31/14; A61K 31/136; A61K 9/00

(52) U.S. Cl. CPC A61L 31/146; A61K 9/00; A61K 31/136
USPC 424/400; 514/658; 428/312.6

(57) ABSTRACT

Treatment of a fungal biofilm on implants is via a combination of drugs and/or via the implant pre-treatment with one drug or via the combination of drugs.

### FIELD OF THE INVENTION

The present invention relates to novel devices, methods and compositions for the treatment or prevention of microbial biofilms, preferably a fungal and/or yeast biofilm, preferably when grown on a medical device, by increasing the susceptibility and sensitivity of said biofilm to antibiotic drugs and/or by imparting a continued highly localised treatment with said antibiotic drugs.

More in particular, said methods and compositions comprise combining an antimicrobial agent, such as an antifungal agent, with an Efg1 inhibitor or antagonist, preferably diclofenac or a derivative thereof, for eradicating, inhibiting or preventing fungal biofilms or fungal biofilm formation in a subject and/or on a solid support surface or other medium susceptible to biofilm formation.

Alternatively, said devices relate to a medical device, particularly an implantable device adapted to release in a controlled manner a substance or composition, particularly a substance or composition capable of eradicating, inhibiting or preventing fungal biofilms or fungal biofilm formation, such as an Efg1 inhibitor and/or an antifungal.

### BACKGROUND OF THE INVENTION AND STATE OF THE ART

### Biofilms & Medical Devices

Biofilms are an alternate mode of microbial growth where cells exist within a complex and highly heterogeneous matrix of extracellular polymers adherent to a surface. Microbial biofilms display decreased susceptibility to antimicrobial agents and elevated resistance to host immune response, often causing chronic infections. Due to their increased resistance to antibiotic (antifungals) agents biofilms have a tendency to become pathogenic.

Due to the increasing number of immunocompromised patients, combined with the advances in medical technology, fungi have emerged as a major cause of infectious disease, with Candida albicans being the major pathogen.

Apart from their existence under free-living or planktonic form, Candida sp. are known to form biofilms upon contact with various surfaces.

C. albicans cells are able to colonize and subsequently form biofilms on surfaces of indwelling medical implants and devices, such as (dental) implants, intravascular and urinary catheters, voice prostheses and heart valves.

The formation of biofilms on such medical devices, particularly fungal/C. albicans biofilms that are resistant to current antibiotics (antifungals), is the major factor responsible for biofilm associated infections, particularly implant infections. In many cases, the implant has to be removed in order to cure the infection.

Implant failure leads to burdensome and costly revision surgery and sometimes severe suffering of the patient.

### Strategies Against (Fungal) Biofilms

Today, there are 4 main classes of established antifungal drugs on the market: (i) the polyenes (e.g. amphotericin B, nystatin, natamycin), (ii) the azoles (e.g. miconazole, fluconazole, itraconazole, voriconazole), (iii) allylamines (e.g. terbinafine), and (iv) echinocandins (e.g. caspofungin).

Of these classes, only the polyenes, azoles and echinocandins are used to treat systemic fungal infections, not the allylamines.

Among echinocandins, Caspofungin disturbs the integrity of the fungal cell wall by inhibiting the β-(1,3)-D-Glucan synthase and appears to lyse sessile cells within C. albicans biofilms. However, Caspofungin doses that are effective against planktonic cells (i.e. 0.05 μM-0.5 μM) do not decrease the metabolic activity of C. albicans biofilm cells.

Fungal biofilms, especially those of the pathogen C. albicans, are a cause of infections associated with medical devices like indwelling intravascular catheters and implants. Such infections are particularly serious because biofilm-associated Candida cells are relatively resistant to a wide spectrum of antifungal drugs, including ROS-inducing antifungal compounds, such as Amphotericin B (AmB) and azoles.

Among the current antifungals in clinical use, only the liposomal formula of amphotericin B and echinocandins have shown unique and consistent in vitro and in vivo activity against C. albicans biofilms.

The prior art disclose several, sometimes conflicting, reports dealing with C. albicans biofilm sensitivity. On the one hand, previous reports have demonstrated that aspirin can inhibit C. albicans biofilm formation in vitro or that cyclo-oxygenase inhibitors can reduce biofilm formation (Alem and Douglas (2004) Antimicrob Agents Chemother 48:41-7) or inhibit yeast-to-hyphae transition in vitro. However, inter-study variations are observed in vitro, which highlight the differences in susceptibilities of Candida biofilms, which can be strongly influenced by different setups of the experiments in vitro, including the type of device and composition of the medium and pH (Kucharíková et al. (2011) J Med Microbiol 60:1261-9).

Furthermore, although hyphal formation is pivotal for biofilm development in C. albicans, the sessile lifestyle associated with adherent cells is believed to confer antifungal resistance, regardless of coherent biofilm formation (Ramage et al. (2009) Crit. Rev Microbiol, 35, 340-55.

Therefore, an in vitro activity against planktonic or biofilm cells does not equate to an in vivo antifungal effect, especially in the case of the treatment of an established biofilm (and/or of sessile cells), such as on the surface of medical devices or implants.

In addition, all the currently marketed antifungal drugs have major drawbacks, including no broad-spectrum activity, no per oral absorption, side-effects, low antifungal activity, no fungicidal activity, drug-drug interactions and/or high costs. In the case of biofilm treatments or of treatments of sessile cells, these drawbacks become prohibiting.

Also, antibiotics (antimycotics) that are active against microbial biofilms often result in only partial killing of the biofilm cells, even when applied at high doses, leaving a subpopulation of the biofilm cells alive, the so called persisters. Persisters are antibiotic-tolerant cells that survive treatments with high antibiotic concentrations. Because they start growing again when the antibiotic pressure drops, persisters are considered as one of the most important reasons for the recurrence of biofilm-associated infections.

Accordingly, there is a clear need for effective strategies to prevent and to eliminate deleterious biofilms, in particular fungal or yeast biofilms associated with the surface of medical devices, such as implants.

### SUMMARY OF THE INVENTION

The present invention tackles the increasing problems of biofilms, in particular of fungal biofilms on different surfaces within the human body and which escape conventional antibiotic treatment. The present invention provides devices, methods and compositions with improved anti-biofilm properties by enhancing the efficacy of antibiotic and/or antimycotic drugs, such as by increasing the susceptibility and sensitivity of biofilms, particularly of fungal biofilms to said drugs and/or by a continued, highly localised treatment with said drugs.

A first aspect of the invention is a medicament selected from the group consisting of antibiotics, antifungal, antiviral, anti-inflammatory, analgesic, antihypertensive and a combination thereof (preferably antifungal and/or anti-inflammatory) for use in the treatment or in the prevention in patient of a microbial (fungal and/or comprising or consisting essentially of Candida sp., such as C. albicans) infection in the form of a biofilm, wherein the said patient has been pre-implanted with a biocompatible device comprising a surface susceptible of microbial biofilm formation and wherein this device comprises this medicament (preferably an antifungal and/or anti inflammatory) and wherein this medicament is released upon this device surface.

Preferably, the (pre-implanted) device comprises and/or is connected to a reservoir comprising this medicament.

Preferably, the (pre-implanted) device comprises one or several layer of pores having a size comprised between 0.3 nm and 50 μm (or even more).

More preferably, the (pre-implanted) device comprises a (first) (macro)porous layer in ceramic materials, in metals and/or in metal alloys (advantageously in titanium and/or in titanium derivatives), this (macro)porous layer having a pore size between 50 nm and 1000 μm, preferably between 200 nm and 20 μm, more preferably between 500 nm and 10 μm, still more preferably between 1 μm and 2 μm.

More preferably, the (pre-implanted) device comprises a (second) (meso- and/or micro-)porous layer (advantageously) in silicium oxide and derivatives thereof, this (second (meso- and/or micro-)porous layer having a size comprised between 0.3 nm and 300 nm, preferably between 1 nm and 100 nm, more preferably between 2 nm and 30 nm.

More preferably, the medicament for use in a patient having this (pre-implanted) device comprises (or consists (essentially) of) an anti-inflamatory agent and/or an Efg1 inhibitor, still more preferably diclofenac or a derivative thereof.

More preferably, the medicament for use in a patient having this (pre-implanted) device (further) comprises an antifungal agent selected from the group consisting of polyenes, azoles, allylamines, echinochandins and piperazine-1-carboxamidine derivatives.

More preferably, the medicament for use in a patient having this (pre-implanted) device (further) comprises a biologically active agent selected from the group consisting of hormones, cytokines, growth factors, antibodies, immune-suppressive, antineoplastic agents and combination thereof.

A related aspect of the present invention is (a pharmaceutical composition of) an Efg1 inhibitor and an antifungal agent selected from the group consisting of polyenes, azoles, allylamines, echinochandins and piperazine-1-carboxamidine derivatives for use in the treatment (or even in the prevention) of a fungal infection in a patient, wherein this fungal infection is in the form of a biofilm (wherein preferably the biofilm consists (essentially of) Candida sp. cells).

Preferably the Efg1 inhibitor (of this pharmaceutical composition) is diclofenac or a derivative thereof.

Advantageously, the amount of this antifungal agent is the amount effective against the fungal cell infection in a planktonic form (i.e. an about 5-fold to an about 10-fold lower amount than the amount effective against this fungal infection in biofilm form).

More preferably, this antifungal agent is selected from the group consisting of caspofungin, miconazole and amphotericin and wherein still more preferably the caspofungin amount is comprised between 0.5 mg/kg and 15 mg/kg body weight of a patient, the miconazole amount is comprised between 2 mg/kg and 20 mg/kg body weight of a patient and the amphotericin amount is comprised between 0.05 mg/kg and 1.5 mg/kg body weight of a patient.

Another related aspect of the present invention is an implant and/or biocompatible device comprising one or several layer of pores having a size comprised between 0.3 nm and 1000 μm, wherein this implant or device is connected to a reservoir comprising a pharmaceutical composition and/or wherein this implant or device comprises a pharmaceutical composition and wherein this pharmaceutical composition is selected from the group consisting of antibiotics, antifungal agents, sensitizing agents, anti-inflammatory agents, analgesic agents and a combination thereof.

Preferably, this implant and/or biocompatible device comprises a first layer of (macro-)pores having a size above 50 nm.

More preferably, this first layer of pores is in ceramic materials, in metals (oxide) and/or in metal alloys (preferably in titanium and/or in titanium derivatives), still more preferably, the first layer consists essentially of a metal or metal alloy, such as titanium or a titanium alloy.

More preferably, this first layer of pores has a diameter comprised between 100 nm and 20 μm, still more preferably between 1 μm and 2 μm.

More preferably, this implant and/or biocompatible device comprises a second layer of pores having a size comprised between 0.3 nm and 300 nm, still more preferably between 1 nm and 100 nm.

Preferably, this second layer of (meso- and/or micro-)pores is in (amorphous) silicium oxide and/or in silicium oxide derivatives (or in compounds comprising more than 20%, 30%, 40%, 50%, 60%, 70%, 80% (w:w) or even more of silicium oxide).

More preferably, in this implant, the pharmaceutical composition comprises an anti-inflammatory agent and/or an Efg1 inhibitor, still more preferably diclofenac or a derivative thereof.

More preferably, in this implant, the pharmaceutical composition (further) comprises an antifungal.

Advantageously, this implant and/or biocompatible device is (a surface of) a medical device selected from the group consisting of catheters, implants, prostheses, stents, surgical plates, valves or pins, artificial joints, pacemakers, contacts lenses and bio-implants.

Another related aspect of the present invention is an antifungal agent for use in the treatment or in the prevention of an infectious (fungal such as consisting (essentially of) Candida sp) disease in a patient, wherein this patient has been pre-implanted with a solid support surface (implant and/or device according to the present invention) coated with an Efg1 inhibitor.

The preferred Efg1 inhibitor is diclofenac or a derivative thereof.

Preferably, the antifungal agent (used to treat or to prevent the infectious disease) is selected from the group consisting of polyenes, azoles, allylamines or echinochandins and piperazine-1-carboxamidine derivatives, such as an antifungal selected from the group consisting of caspofungin, miconazole and amphotericin.

Advantageously, the amount of this antifungal agent is calculated as effective against the fungal cell infection in a planktonic form.

Another related aspect of the present invention is an Efg1 inhibitor (preferably diclofenac or a derivative thereof) for use in increasing in a patient, suffering of a fungal infection, the amount of an antifungal agent inside fungal cells, wherein the said fungal cells are in the form of a fungal biofilm.

Preferably, the antifungal agent is selected from the group consisting of polyenes, azoles, allylamines or echinochandins and piperazine-1-carboxamidine derivatives, being more preferably selected from the group consisting of caspofungin, miconazole and amphotericin.

Another related aspect of the present invention is the non-therapeutical use of an Efg1 inhibitor (preferably diclofenac or a derivative thereof) for increasing the intracellular amount of an antifungal agent inside fungal cells, wherein the said fungal cells are in the form of a fungal biofilm (consisting (essentially of) Candida sp cells).

Preferably, the antifungal agent is selected from the group consisting of polyenes, azoles, allylamines or echinochandins and piperazine-1-carboxamidine derivatives, more preferably is selected from the group consisting of caspofungin, miconazole and amphotericin.

Another related aspect of the present invention is a method for the treatment and/or for the prevention of a fungal biofilm upon a (solid support) surface comprising the steps of:
treating (coating) the surface with an efficient amount of an Efg1 inhibitor;
introducing the said coated surface in a mammal body, including a human patient body;
perfusing the said introduced coated surface and/or the said mammal, including said human, with an efficient amount of an antifungal agent.

Advantageously the efficient amount of the antifungal agent is the amount efficient against the fungal cells infection in a planktonic form.

Another related aspect of the present invention is a method for reducing, destroying, eradicating, inhibiting or preventing fungal biofilms or fungal biofilm formation, wherein a (solid support) surface or medium susceptible to biofilm formation is treated with an Efg1 inhibitor and at least one antifungal agent.

### BRIEF DESCRIPTION OF THE FIGURES

FIG. 1 represents the sensitivity of C. albicans biofilms to miconazole. Biofilms of CAF2 (isogenic WT) (circles), Δefg1 (squares) and the EFG1 reintegrant Δefg1(EFG1) (triangles) were treated with miconazole (0-250 μg/ml).

FIG. 2 shows the Intracellular miconazole accumulation in C. albicans biofilms. Biofilms of CAF2 (isogenic WT, white bars) and Δefg1 (grey bars) were treated with different concentrations of miconazole. The intracellular miconazole concentration was determined by quantitative HPLC. *p<0.05.

FIG. 3 shows the effect of diclofenac on the sensitivity of C. albicans biofilms to miconazole. Biofilms of CAF2 (isogenic WT) (circles), CAF2 grown in the prescence of 500 μg/ml diclofenac (triangles) and Δefg1 (squares) were treated with miconazole (0-250 μg/ml). % remaining biofilm was calculated relative to control treatment (without miconazole, in the absence or presence of diclofenac).

FIG. 4 shows the effect of diclofenac on the sensitivity of C. albicans biofilms to miconazole. Biofilms of CAF2 (isogenic WT) (squares), CAF2 grown in the prescence of 2 mM diclofenac (open triangles) were treated with miconazole (0-500 μM). The viable biofim cells were determined using the CellTiter-Blue staining and % fluorescence was calculated relative to control treatment (without miconazole, in the absence or presence of diclofenac), as a measure for biofilm cell viability.

FIG. 5 shows the intracellular miconazole levels in C. albicans biofilms, normalized to the total number of cells in the biofilms. C. albicans biofilms were grown in the presence (gray bars) or absence (black bars) of 500 μg/ml diclofenac, after which they were treated with various concentrations of miconazole. ***, p<0.001

FIG. 6 shows the effect of diclofenac on the sensitivity of C. albicans biofilms to Amphotericin B. Biofilms of CAF2 (isogenic WT) (diamonds), CAF2 grown in the prescence of 500 μg/ml diclofenac (squares) and Δefg1 (triangles) were treated with 2.5-10 μg/ml Amphotericin B. The remaining living biofilm cells were assessed by CFU determination and expressed as % survival relative to DMSO control (without AmB, in the presence or absence of diclofenac).

FIG. 7 shows the effect of diclofenac on the sensitivity of C. albicans biofilms to caspofungin. Biofilms of CAF2 (isogenic WT) (Diamonds), CAF2 grown in the prescence of 2 mM diclofenac (open circles) were treated with caspofungin (0-125 μM). The viable biofim cells were determined using the CellTiter-Blue staining and % fluorescence was calculated relative to control treatment (without caspofungin, in the absence or presence of diclofenac), as a measure for biofilm cell viability.

FIG. 8 shows the sensitivity of diclofenac-treated and -untreated Candida albicans biofilms (grown on catheters) to caspofungin in vitro. Biofilms of C. albicans wild type CAF2 were grown in the absence (control) or presence of 2 mM diclofenac and treated with or without 125 μM caspofungin. After incubation for 24 h, biofilm cells were quantified by determination of colony forming units (CFUs). **p<0.01.

FIG. 9 shows the sensitivity of C. albicans biofilms grown on diclofenac-soaked or untreated catheters and treated with or without 125 μM caspofungin. After incubation for 24 h, biofilm cells were quantified by determination of colony forming units (CFUs). *p<0.05.

FIG. 10 shows the sensitivity of diclofenac-treated and -untreated Candida albicans biofilms, grown on implanted catheter fragments, to caspofungin in vivo. Catheters were inoculated with C. albicans CAF2 cells prior to subcutaneous implantation in rats. Rats received either vehicle control or 3 mg/kg caspofungin treatment with or without 3 mg/kg diclofenac. The number of C. albicans cells recovered from implanted catheter fragments from each tested group are shown. *p<0.05; ***p<0.001.

FIG. 11 represents a schematic view showing a cross section of an implantable device with injection port in which (a) constitutes a fully dense or solid part serving as structural basis for the device, (b) is a port allowing attachment of a syringe, (c) a porous structure suitable for bone tissue attachment or ingrowth and (d) an optional cavity allowing a larger volume of fluid to be injected.

FIG. 12 represents a schematic view of a cross section of a dental implant screw composed of a solid base with incorporated injection port and a porous screw of which the internal pore surface is coated with amorphous mesoporous silica (AMS) which acts as slow release medium and the external screw surface is coated with a biofilm inhibiting coating, particularly a peptide coating.

FIG. 13 represents the slow release properties of two samples SiO₂-deposited porous Ti discs (Sample A & B) via a methylene blue percolation test. The methylene blue concentration at the collector side is expressed relative to that of the feed concentration.

FIG. 14 shows an image (A) and schematic representation (B) of the Swagelok® body in which a Ti disc (and an O-ring fitting) are mounted.

### DETAILED DESCRIPTION OF THE INVENTION

The inventors have developed devices, methods and compositions for reducing, destroying, eradicating, inhibiting (or preventing) biofilms (or biofilm formation), preferably of fungal origin, which escape conventional antibiotic (antifungal) treatment, in a subject and/or on a solid support surface or other medium susceptible to biofilm formation.

Particularly, the inventors have developed means for increasing the susceptibility and/or sensitivity of biofilms, particularly of fungal biofilms, to antibiotic and/or antimycotic drugs.

In this respect, the inventors have found that the efficacy of said drugs is enhanced by using a medical device with the intrinsic capacity to inhibit or prevent biofilm formation on the surface of such medical device, thus imparting a highly localised treatment with said drugs, and/or by combining said antibiotic (and/or antifungal) drugs, even at sublethal dosages, with a suitable biofilm sensitizing bio-active agent, particularly an Efg1 inhibitor. Particularly, said medical device allows administering one or more bio-active agents, including but not limited to antibiotics, both antibacterial and antifungal, sensitizing agents, anti-inflammatory agents and/or analgesic agents, through the implant porous structure to the surrounding tissue, resulting in a localized prevention or treatment of complications associated with implant surgery.

When using the devices, methods and composition of the present invention, dosages of antimicrobial agents may be reduced but remain effective in eradicating, inhibiting or reducing biofilms. Particularly, such treatment may be continued during a defined period of treatment and at a constant (localised) dose without antimicrobial pressure drops so that development of resistance, persisters and recurrence of biofilm-associated infections can be prevented.

### DEFINITIONS

As used herein the term “biofilm” refers to a mode of microbial growth comprising sessile cells, usually within a complex and highly heterogeneous matrix of extracellular polymers, and characterized by a reduced sensitivity to antimicrobial agents.

“Reducing”, “inhibiting”, “preventing” or the like in reference to a biofilm or biofilm formation means complete or partial inhibition (more than 50%, preferably more than 90%, still more preferably more than 95% or even more than 99%) of biofilm (in the term of number of remaining cells) formation and/or development. Further, inhibition may be permanent or temporary. In terms of temporary inhibition, biofilm formation and/or development may be inhibited for a time sufficient to produce the desired effect (for instance at least 5 days, preferably at least 10 days. Preferably, the inhibition of biofilm is complete and/or permanent (no persisters).

In the context of the present invention, biofilms can contain single species (e.g. a fungi/yeast such as C. albicans) or multiple species microorganisms (such as C. albicans and other microorganisms, preferably yeasts and/or fungi or even prokaryotes).

The present invention is advantageously related to the biofilms that are associated with microbial infection (e.g., burns, wounds or skin ulcers) or a disease condition including, without limitation, dental caries, periodontal disease, prostatitis, osteomyelitis, septic arthritis, and cystic fibrosis.

Advantageously, the biofilm of the present invention is a fungal and/or yeast) biofilm, more preferably a Candida species (e.g. C. albicans, C. glabrata, C. krusei) biofilm, an Aspergillus species (e.g. A. flavus, A. fumigatus, A. clavatus) biofilm or a Fusarium species (e.g. F. oxysporum, F. culmorum) biofilm, most preferably a Candida albicans biofilm and can be associated with fungal infection on (the surface of) medical devices like indwelling intravascular catheters and implants and in the oral cavity (e.g. on dental implants).

The present invention preferably relates to biofilms associated with a surface, e.g., a solid support surface.

Such surface can be the surface of any (industrial) structure or the surface of any structure in animals or humans.

For example, such surface can be any epithelial surface, mucosal surface, or any host surface associated with microbial infection, e.g., persistent and chronic microbial infections.

Preferably, the surface is a surface of a bio-device in animals or humans, including without limitation, bio-implants such as bone prostheses, dental implants, heart valves, pacemakers and indwelling catheters.

In a preferred aspect, this microbial and/or fungal biofilm (e.g. consisting (essentially) of C. albicans) is associated with the oral cavity, including the surface of dental implants or speech prostheses.

In addition to surfaces associated with biofilm formation in a biological environment, the surfaces can also be any surface associated with industrial biofilm formation. For example, the surfaces being treated can be any surface associated with biofouling of pipelines, heat exchangers, air filtering devices, or contamination of computer chips or water-lines in surgical units like those associated with dental hand-pieces.

The term “microporous material” as used herein is in the meaning of solids, preferably solid silica, that contain pores with free diameters of molecular dimensions. The upper limit of the micropore diameter range according to IUPAC is 2 nm.

The term “mesoporous material” as used herein is in the meaning of solids that contain pores with free diameters of 2-50 nm (preferably, between 3 nm and 30 nm, more preferably between 4 nm and 25 nm). A smaller diameter of the mesoporous (and of microporous) material was found beneficial for the (in vivo) controlled release of the drug.

The term “macroporous material” as used herein is in the meaning of solids that contain pores with free diameters above 50 nm (for instance between 100 nm and 1000 μm, preferably between 200 nm and 20 μm, more preferably between 500 nm and 10 μm, still more preferably between (about) 1 μm and (about) 2 μm). A bigger diameter was found beneficial for allowing the surrounding tissue to invade parts of the implant.

Micropores are conveniently subdivided into ultramicropores narrower than 1.5 nm, and supermicropores with free diameters from 1.5 to 2 nm. Of the porous substances, those having uniform channels, such as zeolite, are defined as molecular sieves.

The term “sol” as used in this application means a colloid that has a continuous liquid phase (e.g. an aqueous phase) in which a solid with a particle size in the micrometer range or smaller is suspended. Sol is synonymous to colloidal suspension.

The term “gel” as used herein refers to a material consisting of continuous solid and liquid phases of colloidal dimensions.

The term “sol-gel” as used herein means a gel derived from a sol, either by polymerising the sol into an interconnected solid matrix, or by destabilising the individual particles of a colloidal sol by means of an external agent. Sol-gel materials may be produced in a wide range of compositions (mostly oxides) in various forms, including powders, fibres, coatings, thin films, monoliths, composites, and porous membranes. In general, the sol-gel process involves the transition of a colloidal suspension system into a “gel” phase exhibiting a significantly higher viscosity.

The term “amorphous” or “amorphous structure” as used herein means without an apparent long range order of the atom positions, therefore lacking crystallinity.

The term “controlled release” as used herein refers to a relatively slow or delayed or prolonged release of a bio-active compound from a device in its environment. Particularly, an 80% release of the bio-active compound into an aqueous fluid at a pH between 1.0 and 8.0 is only obtained when a period of time of at least 30 minutes, preferably at least 60 min, at least 24 hours, or at least 48 hours has passed, even more preferably when a period of time lasting several hours, days, weeks or even months has passed (i.e. 20% (or more) of the bio-active compound remains in the device after at least 30, 60 min, 24, 48 h or even several days).

A first object of the present invention provides methods and compositions comprising combining an antimicrobial (antibiotic) agent, such as an antifungal agent, with an Efg1 inhibitor or antagonist, for reducing, destroying, eradicating, inhibiting or preventing (fungal) biofilms or (fungal) biofilm formation, particularly on a solid support surface or other medium susceptible to biofilm formation.

The inventors have found that the transcription factor Efg1 plays a role in antifungal tolerance of fungal and/or yeast (Candida, such as C. albicans) biofilms (and/or of sessile cells), especially in vivo.

Inhibition of Efg1 function or expression, such as by diclofenac (or using mutated isogenic strains) is shown by the inventors to increase (especially in vivo) the susceptibility and sensitivity of biofilms (and/or of sessile cells) of microorganisms, in particular fungal species, and/or yeast (Candida) to antimicrobial or antifungal drugs, even in situations where Efg1 inhibition does not affect (reduce) the biofilm structure, nor prevents biofilm formation.

In contrast to the Efg1 inhibitor (diclofenac) or the antifungal(s) (miconazole or amphotericin or caspofungin) alone, the combination of both synergizes (especially in vivo) against these microorganisms (in the form of biofilms (and/or of sessile cells)) in sharply reducing the number of microbial cells, in particular fungal species and/or yeast (Candida) or of frequency of infections.

Combining substances that inhibit Efg1 or reduce Efg1 expression levels with one or more conventional antifungal agent(s) is proven by the inventors to be useful to treat, prevent or reduce a fungal infection in the form of (organized into) biofilms in a mammal subject, preferably a human patient.

In addition, because of the sensitizing effect the Efg1 inhibitor (diclofenac), combination therapy comprising an Efg1 inhibitor (diclofenac) and an (one or more) antimicrobial or antifungal compound(s) allows reducing the antimicrobial dose or even applying an antimicrobial concentration which would be sub-lethal (for a microorganism in the form of a biofilm and/or of sessile cells) in the absence of the Efg1 inhibitor.

Particularly, the present invention shows that the antifungal activity of antimycotics, particularly echinocandins, such as caspofungin (against a microorganism, in particular a fungal species, and/or yeast such as Candida, in the form of a biofilm and/or sessile cells), can be improved by combining the antifungal compound(s) with this Efg1 inhibitor, preferably diclofenac, and thus concomitantly resulting in a reduction of its minimal inhibitory concentration (MIC) against a microorganism infection, especially fungal infection and/or Candida (C. albicans) in the form of biofilms.

The inventors have further found that Efg1 inhibitor allows for the use of an (one or more) antifungal agent(s) against a fungal species, and/or yeast, such as Candida infection in the form of a biofilm at a reduced amount, which is the amount effective against this (fungal) infection in planktonic form (non sessile, non forming a biofilm).

Accordingly, the present invention also relates to the combined use of an inhibitor of Efg1 function or expression and of an (one or more) antimicrobial or antifungal agent(s) for (use in) the treatment or prevention of (fungal infections in the form of) biofilms, particularly fungal and/or yeast biofilms, such as Candida biofilms.

“Efg1 inhibitor” in the context of the present invention refers to a material that act on another biopolymer, such as an Efg1 polypeptide or an EFG1 polynucleotide (such as SEQ. ID. NO. 1), which can inhibit the activity or expression of said Efg1 transcription factor or an Efg1 ortholog in other (fungal) species.

This Efg1 inhibitor may act as a factor, as an inhibitor, as antibodies, as tags, as a competitive inhibitor, or alternatively have antagonistic activity vs. the function of Efg1.

This Efg1 inhibiting material or agents include small molecules, natural products, oligonucleotides (antisense or aptamers), antigenic therapeutics, small interfering RNAs (or RNA interference in general) and antibodies.

Preferably, this Efg1 inhibitor is a NSAID (Non-Steroidal Anti Inflammatory Drug), more preferably is diclofenac or a derivative thereof, such as the compounds described in U.S. Pat. No. 3,558,690.

Preferably the antimicrobial agent is an antifungal agent.

Suitable antifungal agents include the polyenes (e.g. amphotericin B, nystatin, natamycin), the azoles (e.g. miconazole, fluconazole, itraconazole, voriconazole), allylamines (e.g. terbinafine), the echinocandins (e.g. caspofungin) or the piperazine-1-carboxamidine derivatives described in WO2010068296.

A preferred aspect relates to a combination therapy of diclofenac (or a derivative thereof) with one or more antifungal drug(s), like miconazole, Amphotericin B or caspofungin (or other conventional antifungal agents) to treat or prevent (Candida) biofilm associated infections (in a mammal subject, preferably a human patient).

Advantageously, the inventors have found that the addition of an Efg1 inhibitor (such as between about 0.5 mg/kg and about 15 mg/kg, preferably between about 1 mg/kg and about 10 mg/kg, more preferably between about 2 mg/kg and about 5 mg/kg, still more preferably of about 3 mg/kg body weight of diclofenac in a patient and/or of between about 100 μg/ml and about 10 mg/ml, preferably between about 200 μg/ml and about 1 mg/ml, more preferably of about 500 μg/ml of diclofenac) allows for the use of caspofungin doses (on a daily basis) of about 0.05 μM to about 0.5 μM and/or of doses of about between about 0.5 μM and about 15 μM, preferably between about 1 mg/kg and about 10 mg/kg, more preferably between about 2 and about 5 or about 3 mg/kg body weight of a patient; or for the use of miconazole doses between about 20 μg/ml and about 250 μg/ml, preferably between about 50 μg/ml and about 200 μg/ml, more preferably between about 60 μg/ml and about 100 μg/ml, more preferably between about 1 mg/kg and about 50 mg/kg, preferably between about 1 mg/kg and about 25 mg/kg, more preferably between about 2 mg/kg and about 20 mg/kg, still more preferably of about 5 to 10 mg/kg body weight of miconazole in a patient; or for the use of amphotericin B doses between about 1 μg/ml and about 10 μg/ml, preferably between about 2 μg/ml and about 5 μg/ml, more preferably of about 3 μg/ml of amphotericin, more preferably between about 0.05 mg/kg and about 1.5 mg/kg, preferably between about 0.1 mg/kg and about 1.0 mg/kg, more preferably between about 0.2 mg/kg and about 0.5 mg/kg, still more preferably of about 0.3 mg/kg body weight of Amphotericin in a patient.

Another related aspect is the combination of a medical device, particularly an implant, and a pharmaceutical composition comprising one of several medicament(s) for preventing or suppressing biofilms in a patient (preferably a mammal, more preferably a human).

Another preferred aspect relates to the preparation (e.g by coating) of a solid support surface, such as a medical device, like implants, plastics or (subcutaneous) catheters, with the addition of a sufficient amount (or dose) of diclofenac upon this solid support surface (or inside the solid support), for the elimination (reducing, destroying, eradicating, inhibiting or preventing) of Candida biofilms in combination with conventional antifungal therapy (addition of one or more conventional antifungal compound(s) at the dose effective against the planktonic form).

The inventors have found that biofilms of C. albicans Δefg1, lacking the transcription factor Efg1, showed increased antifungal susceptibility as compared to WT or reintegrant Δefg1(EFG1) biofilms.

Moreover, Δefg1 biofilm cells accumulated more intracellular antifungal (miconazole) than WT biofilm cells, suggesting the presence of an import- or efflux-dependent antifungal (miconazole) tolerance mechanism involving Efg1.

Hence, this transcription factor is demonstrated by the inventors to be a suitable target for future anti biofilm therapy.

The combination of (the non-steroid, anti-inflammatory drug) diclofenac with one or more conventional antifungal agent(s) (particularly, azole(s), polyene(s), or echinocand(s)) (e.g. miconazole, Amphotericin B and Caspofungin) was effective in reducing Candida biofilms.

Accordingly, an aspect of the present invention relates to a method for the treatment or prevention of a microorganism (fungal) infection in the form of a biofilm, wherein the microorganisms within this biofilm, or capable of forming a biofilm, are exposed to an (effective amount of an) Efg1 inhibitor and, before, after or concurrent with the Efg1 inhibitor, exposing the microorganisms within this biofilm or capable of forming a biofilm to at least one antimicrobial agent, preferably one or more antifungal agent(s) (drug(s) or compound(s) (not an Efg1 inhibitor).

The present invention also relates to a method for inhibiting biofilm formation and/or development, wherein a solid support surface or other medium susceptible to biofilm formation is treated (coated) with an Efg1 inhibitor or wherein an Efg1 inhibitor is incorporated in a solid support surface or medium susceptible to biofilm formation, such as incorporated in a controlled release medium or (porous) support, and wherein, concurrently or subsequently, this surface or other medium susceptible to biofilm formation is exposed to an antimicrobial agent.

Possibly, the microorganisms present in the biofilms or capable of forming biofilms may be of a single species or of multiple species and may comprise bacterial or fungal species or both.

Preferably, this biofilm is a fungal biofilm, more preferably a Candida biofilm, comprising C. albicans, C. glabrata and/or C. Krusei and/or consisting essentially of C. albicans.

The term “consisting essentially of C. albicans” refers to a percentage (number of C. albicans cell:total cell) in the biofilm. Preferably the percentage is above 50%, more preferably above 75%, still more preferably above 90% and/or this term refers to the fact that C. albicans is present in an amount (concentration) sufficient to provoke the biofilm.

The present invention also relates to a method for the treatment and/or prevention of a condition (fungal infection) associated with biofilm development, comprising administering to a subject an effective amount of an Efg1 inhibitor and an (one or more) antimicrobial agent(s). Thus, a method of the invention for treating or preventing formation of biofilms (e.g. a fungal infection in the form of a biofilm) may comprise administering to a subject an effective amount of an Efg1 inhibitor together with at least one antimicrobial agent for the treatment or prevention of a biofilm-associated condition (fungal infection) in this subject.

The Efg1 inhibitor and/or the antimicrobial agent may thus be coated onto the surface of or be impregnated in or incorporated in a suitable medical device such as a catheter, stent, prosthesis or other surgical or implantable device. Advantageously, said Efg1 inhibitor and/or the antimicrobial agent is released locally in a slow, controlled and/or continuous manner by said medical device.

According to another aspect of the invention, there is provided a composition for promoting dispersal of, or preventing formation of a microbial biofilm, the composition comprising one or more Efg1 inhibitors, more preferably diclofenac, and at least one antimicrobial agent, preferably an antifungal agent.

It will be readily appreciated by those skilled in the art that according to the methods of the invention each component of the combination may be administered at the same time, or sequentially in any order, or at different times, so as to provide the desired effect.

Alternatively, the components may be formulated together in a single dosage unit as a combination product.

The present invention also relates to compositions for treating and/or preventing a condition (fungal infection) associated with biofilm development.

Typically the compositions provide means for carrying out the methods of the invention.

Advantageously, the methods and compositions of the invention described above find application in a wide range of environments and circumstances. The composition may be an anti-fouling composition, a medical device or component thereof, a coating for a medical device or a pharmaceutical composition.

Preferably, the composition(s) of the invention or one or more components thereof, may (also) be used in coating medical devices, including implantable medical devices, including but not limited to venous catheters, urinary catheters, stents, prostheses such as artificial joints, hearts, heart valves or other organs, pacemakers, surgical plates and pins and contact lenses.

Other medical equipment may also be coated, such as catheters and dialysis equipment.

If either the Efg1 inhibitor or the antimicrobial agent is not coated on said surface, the non-coated component (this Efg1 inhibitor or this antimicrobial agent) can then be administered in an alternative manner.

Methods and compositions of the invention also find application in the management of infectious diseases. For example, a variety of infections associated with (fungal) biofilm formation may be treated with methods and compositions of the invention, such as urinary tract infections, pulmonary infections, dental plaque, dental caries and infections associated with surgical procedures or burns.

Accordingly, compositions of the invention may be formulated as pharmaceutical compositions or form components of, for example, surgical dressings, mouthwash, toothpaste or saline solutions.

Within the context of the present invention, this Efg1 inhibitor and/or this antimicrobial agent may be applied or coated onto, or incorporated in the surface of an object/item of interest (such as to impart a slow or controlled release effect) well in advance of use of this object/item in, or exposure of this object/item to an environment which comprises biofilm-forming microorganisms, or this Efg1 inhibitor and/or this antimicrobial agent may be applied or coated onto, or incorporated in the surface of an object/item of interest immediately before use of that object/item in, or exposure of this object/item to an environment which comprises (or is susceptible to comprise and/or to develop) biofilm-forming microorganisms.

Compositions according to the invention may be in any suitable form. For example a composition of the invention may be formulated as a paint, wax, other coating, emulsion, solution, gel, suspension, beads, powder, granules, pellets, flakes or spray.

As used herein the term “effective amount” includes within its meaning a non-toxic but sufficient amount or concentration of an agent to provide the desired effect. The exact amount/concentration required will vary depending on factors such as the species of microorganism(s) being treated, the extent, severity and/or age of a biofilm being treated, whether the biofilm is surface-associated, the particular agent(s) being administered and the mode of administration and so forth. Thus, it is not possible to specify an exact “effective amount”. However, for any given case, an appropriate “effective amount” may be determined by one of ordinary skill in the art using only routine experimentation.

The antifungal agent(s) (compound(s) or drug(s)) of the present invention are preferably used (or present in composition, including in pharmaceutical composition) at a concentration effective against the planktonic form of the cells, which is a concentration too low to be effective against microorganisms in biofilms (in sessile form).

The inventors have further developed a medical device, particularly a medical implant device, comprising means to increase in its direct environment the efficacy of an anti-biofilm treatment and/or the susceptibility of a biofilm, preferably a fungal and/or yeast biofilm, to said anti-biofilm treatment.

Particularly, said medical implant device is adapted to provide locally a sustained dosage of one or more substances, particularly bioactive agents, including but not limited to an antibiotic agent or a biofilm sensitizing agent, such as an Efg1 antagonist, in order to impart a highly localized treatment or prevention of a biofilm, particularly a fungal or yeast biofilm associated with the surface of said medical device.

Advantageously, the medical implant with improved anti-biofilm properties of the present invention is capable of applying a highly localized, sustained high antibiotic pressure, preferably combined with a sensitizing agent, which is sufficient to kill all microbial cells in a biofilm, particularly a fungal biofilm, leaving no persisters and therefore preventing recurrence of biofilm-associated infections without using potentially toxic dosages of said antibiotic.

Another related aspect of the present invention thus relates to an implantable device with an incorporated injection port and internal cavity or reservoir connected to the environment of said implant through a porous network.

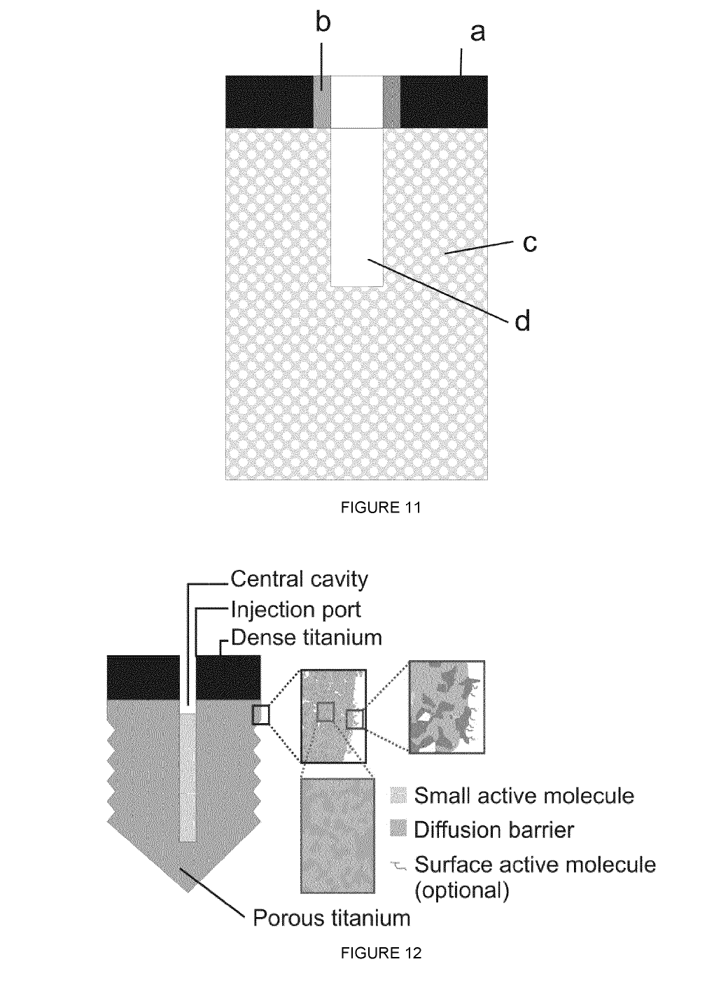

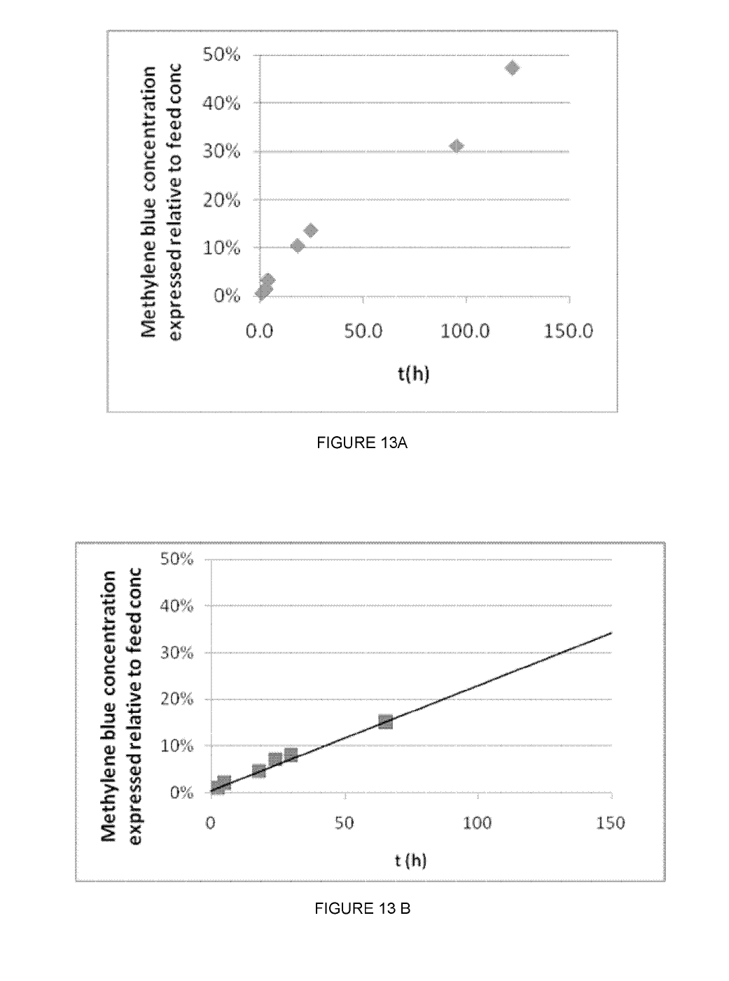

FIGS. 11 and 12 schematically show an implantable device according to present invention. Said porous network is made up of a first macroporous material that is in contact with a second porous material or “a controlled release medium”, preferably comprising smaller pores (such as mesopores or micropores) adapted to form a diffusion barrier for a substance and/or to absorb and release said substance, such as a bioactive agent, from said internal cavity or reservoir in a controlled manner. Said injection port allows supplying various bioactive agents, including but not limited to antibiotics, both antibacterial and antifungal, (biofilm) sensitizing agents, anti-inflammatory agents and/or analgesic agents, to the porous part of the implant and/or the surrounding tissue prior to, during and/or after implantation. Advantageously, prior to or upon the development of infections after full or partial implant fixation, the implant can be filled or injected with a suitable bioactive agent, such as an antibiotic, providing localized treatment or prevention of the infection and thus preventing the need for implant extraction and replacement. Furthermore, the outer surface of the implantable device can be treated or coated with a bioactive agent using state-of-the-art or yet to be developed techniques, such as bioactive molecules which stimulate bone attachment or which inhibit, prevent or reduce microbial attachment or biofilm formation, or a combination thereof. Advantageously, the combination of a biocidal or sensitizing coating on the implant combined with a highly localised treatment after implantation maximizes prevention of (fungal) biofilm formation.

The implantable device of the present invention thus typically comprises (i) a solid or dense structural element; (ii) an injection port incorporated in said solid or dense structural element; (iii) a first porous element, comprising or forming a macroporous network and acting as a macroporous backbone, which is attached to or bonded with said solid element and which can function as a reservoir of substances, molecules or agents, particularly bioactive molecules. Preferably, said implantable device further comprises (iv) a second porous material or controlled release medium in contact with and/or embedded in said (first) porous element, whereby said second porous material comprises smaller pores than said (macro)porous element and is adapted to act as absorbent and/or diffusion resistor or retarder of said substances, such as bioactive molecules that are incorporated in said implantable device.

In addition to the reservoir function of said (first) porous element, the implant may optionally further comprise (v) a cavity able to contain an injected fluid comprising said substances, molecules or agents, particularly bioactive agents. This cavity (d) may be located in the solid element of the implant, being connected with both said injection port and the porous element, but can extend into said porous element as well. The cavity, like the porous element, thus functions as a reservoir whereby a fluid with such agents, molecules or compounds, preferably bioactive agents, are stored in said reservoir, and are subsequently released via diffusion through the porous network (comprising macropores, mesopores and/or micropores) into the tissue environment of the implant. Typically, said cavity allows for a larger volume to be injected.

Said solid or dense element (a) functions as the structural basis of the implant device and typically contains the injection port (b). Said injection port may be adapted to expose part of the porous element that is normally covered by said solid element. It is understood that said solid element, more specifically the injection port, is to remain accessible after implantation. Said injection port may be adapted to easily introduce a fluid into said porous element. Particularly, said injection port may comprise a means to attach an injection device, particularly the proximate end of an injection device, for instance a syringe, for easy refilling of said implant.

Advantageously, said incorporated injection port imparts the ability to administer a bioactive compound, particularly medication, post-surgery and allows easy modification of the treatment by changing the bioactive agents, such as an antibiotic, present in said cavity or reservoir, in the case of e.g. resistant microbial organisms or biofilms or if previously used medication remains ineffective.

Preferably, said porous element comprises a first porous material, particularly a macroporous material, wherein said macropores form a macroporous network that is in contact with the tissue environment of the implantable device and the central cavity or reservoir of the implant device. Typically, said macroporous network functions as the structural basis in which a second porous material is embedded or on which a second porous material is added.

Preferably, the porous element of the implantable device of the present invention is made of an inert, biocompatible material, more preferably said porous element comprises a macroporous titanium or a macroporous titanium alloy, forming a macroporous network or layer that acts as the delivery system of a bioactive agent from the implant to the surrounding tissue. Preferred pore size of said macroporous element is at least 100 nm, more preferably pore sizes range from 200 nm to 1000 μm, more preferably range from 500 nm to 100 μm, even more preferably range 1 μm to 50 μm, such as from 1 μm to 20 μm or 1 to 10 μm or even 1 to 5 μm and 1 to 2 μm.

The porous element of the implantable device can be produced using any of the techniques available in the current state of the art. Said techniques include the use of sacrificial pore templates in powder metallurgical processes, the use of the sponge replication technique, emulsion templating of porous structures, rapid prototyping or additive manufacturing techniques such as selective laser sintering (SLS), selective lased melting (SLM) or electron beam deposition (EBD) or plasma spraying techniques, the partial sintering of metal powder or metal bead compacts or dehydrogenation of metal hydride powder compacts followed by partial sintering [e.g. WO2007000310].

Advantageously, the macropores of said porous element are in contact with a second porous material. Said second porous material (layer) has a smaller pore structure than the macroporous element and comprises small macropores, mesopores and micropores or combinations thereof. Said second porous material may be embedded in said macroporous element, thus partially or completely filling said macropores of the porous element. Alternatively, said second porous material may be coated onto or bonded with said macroporous element, either or both at the outside of said porous element or at the central cavity (if present). Such porous material embedded in or outside the macropores of a porous network have preferably a pore size in the range of 1 nm to 220 nm (or even from 1 to 300 nm), yet more preferably ranging from 2 to 100 nm, yet even more preferably ranging from 3 to 30 nm, such as ranging from 4 to 25 nm or even from 5 nm to 10 nm.

An optional additional feature of the implant of the present invention is that the porous network (layer) is foreseen with a zone of smaller pores in said network (layer) of pores adapted to shield against micro-organisms so that they cannot enter the implant reservoir, the implant central cavity or at least part of the network of pores. Alternatively, the implantable device is composed of a material with a functional gradient in porosity and or pore size. The resulting device can be considered to be fully dense at one side while a porosity suitable for implant purposes is achieved in another part of the device.

Advantageously, said porous element (layer) allows implant fixation by bone attachment and/or bone ingrowth. More particularly, the implantable device comprises at least in part, preferably at the implant site, a porous surface, coating or layer that comprises a certain amount of interconnected porosity with adequate pore sizes to allow a sufficient tissue ingrowth, for instance bone ingrowth, so that a firm mechanical anchorage can be established.

Osseointegration of an implantable device can also be improved by coating the implant material with osseoinductive peptides or proteins, such as growth factors or adhesive proteins, or otherwise incorporating said bone growth enhancing bioactive agents in the implant.

Thus, an additional feature that optionally is comprised in the above described implants is a coating of the outer surface of such implantable device at least in part by bioactive molecules, which can stimulate attachment of bone cells and bone growth around the implant, prevent fungal or bacterial attachment and the formation of biofilms or a combination of both.

Functionalisation by peptides is reported to be feasible by simple immersion of the metallic substrate (implant) in a bioactive peptide-containing solution, by means of covalent bonding to surface hydroxide groups or by incorporation of the peptides into a carrier material such as hydrogels. Said bioactive peptides or signalling proteins can also be placed onto the implant surface by means of electrophoretic deposition (EPD). For instance selected peptides having anti-biofilm properties or promoting osseointegration can be manipulated by means of unbalanced alternating fields. This technique is commonly referred to as alternating current electrophoretic deposition (AC-EPD) [WO2010034826]. This technique enables electrophoresis and subsequent deposition of charged entities while suppressing electrochemical denaturation of solvents and deposited entities, rendering it possible to generate non-releasing coatings of the selected molecules which retain their biological activity.

The implantable device of the present invention may be made of any material suitable to be used in implants or prosthesis, such as metal alloys, ceramic materials (e.g. aluminium oxide or zirconium oxide), high-grade plastics or polymeric materials, as is well known by the person skilled in the art. Typically, said materials are biocompatible, i.e. they can function in the body without creating either a local or a systemic rejection response or other deleterious effect, and they are resistant to bio-erosion, corrosion, degradation and wear, thus retaining their strength and shape for a long time.

Particularly, the implantable device of the present invention may comprise a metal of the group consisting of Ti, Zr, Mg, Hf, Ta, Nd, Nb, Mn, Mo, Al, Cr and Co, or alloys of these elements. More preferably, the implantable device of the present invention comprises Ti, even more preferably is made from ASTM GRADE 1, 2, 3 or 4 Unalloyed Titanium. More preferably, the surface of said implantable device may be oxidized to titanium oxide or dioxide, which in its turn may be further cationic or anionic doped, as is known by the person skilled in the art.

The macroporous network of the porous element is, at least in part or in distinct zones or layers, in contact with a second porous material with smaller macropores, mesopores and/or micropores. Said second mesoporous and/or microporous material may be embedded in said macroporous network or may form a layer or zone surrounding or outside the macroporous network and may thus acts as a diffusion barrier (through which substances, compounds or molecules, particularly bioactive molecules or therapeutic compounds elutes through diffusion) or as a barrier to shield the implant against invasion of microorganisms. More in particular, the rate at which said drugs or compositions are released is controlled through said second material with smaller pores located in the implant's macroporous network.

Preferably, said second porous material in contact with the macroporous layer of said implant is a non-bio-erodible (not disintegrating within a certain period of time by the action of body fluids and/or metabolic activity) porous material, more preferably is a porous oxide (which, in case the macroporous layer is made from an oxide, is typically different from the oxide of the macroporous layer), even more preferably is a silicate based nanoporous material. In the context of the present invention the term “silicate based nanoporous material” refers to porous material with a matrix based on silicon oxide with pore diameter less than 300 nm, preferably of less than 100 nm. The voids between the linked atoms have a free volume larger than a sphere with a 0.25 nm diameter. For pore shapes deviating from the cylinder, the above ranges of diameter of micropores and mesopores refer to equivalent cylindrical pores. Said porous oxides, preferably silicate based nano- or meso-porous material can be either amorphous, ordered or crystalline. Said porous oxides, preferably silicate based porous material can be mesoporous or microporous (or overlapping). Ordered microporous and mesoporous materials can be described in terms of a host structure, which defines a pore structure, which may contain guest species.

Also, current production processes allow to customize pore size and pore size distribution. This way, the release rate of a biologically active compound present in the porous network can be easily controlled. Such porous material embedded in or outside the macropores of a porous network have preferably a pore size in the range of 1 nm to 220 nm, yet more preferably ranging from 1 to 100 nm, yet even more preferably ranging from 1.5 to 30 nm, such as ranging from 2 to 20 nm, 3 to 15 nm or 4 to 10 nm. A narrow pore size distribution is preferred.

The open meso- and/or microporosity of such (amorphous, ordered or crystalline) materials makes them suitable as potential matrices for adsorption and subsequent delayed release of a variety of substances, molecules or compounds, particularly bioactive agents. Also, the diffusion of molecules inside a microporous solid is much slower than inside a mesoporous material, resulting in significantly smaller release rates for the former material. Preferably, said second porous material is an amorphous micro- or mesoporous silica material with pore size ranging from about 1 nm to about 20 nm.

Alternatively, amorphous microporous silica, (less preferably amorphous titania, amorphous zirconia and amorphous alumina) with a narrow monomodal pore-size distribution and a pore size maximum below 1 nm may be chosen as second porous material in contact with the macroporous element. Synthesis methods to prepare such microporous materials are known in the art, e.g. a sol-gel technique comprising polymerisation under acidic conditions.

Microporous amorphous, non-ceramic glasses comprise a matrix of mixed metal oxides, may also be used as second porous material with a narrow pore size distribution in contact with said porous element. Typically, about 90% of the pores of such material have an effective diameter below 3 nm, such as from 0.3 to 1.2 nm (EP812305, EP590714).

Said second porous material may also be an ordered and/or crystalline micro- or mesoporous silica, such as a zeolite or molecular sieve. In the context of the present invention the term “zeolite” refers to a crystalline micro- or mesoporous material comprising coordination polyhedra formed only of silicon, aluminum and oxygen as well as non-aluminosilicate analogs of microporous crystals such as pure silicates, titanosilicates, silicoaluminophosphates and borosilicates, ferrosilicates, germanosilicates and gallosilicates, that exhibit the characteristic molecular-sieving properties similarly to zeolites. A publication entitled “Atlas of Zeolite Structure Types”, 5th Revised Edition (2001) by authors W. M. Meier, D. H. Olson and Ch. Baerlocher, (or http://izac.ethz.ch/fmi/xsl/IZA-SC/ft.xsl) is a good source of the known zeolites and zeolite-like materials, and their different framework types.

Said zeolite may be a crystalline porous material with parallel pores. Zeolite crystals can be grown in situ in the macroporous element. Zeolite synthesis methods are well known by the person skilled in the art. Said zeolite may be grown in situ by a hydrothermal gel method. The synthesis gel typically comprises at least one or more of the following: a source of silica, a source of aluminium, a source of phosphorus and an organic molecule acting as molecular template which is incorporated in the pores of the zeolite during synthesis and which can subsequently be removed by calcinations.

Preferably, said second porous material is a (long range) ordered mesoporous silica based material, having a structure in which mesopores uniform in size are arranged regularly while the constituent atoms show an arrangement similar to that of amorphous silica. These ordered mesoporous materials have the advantage that their pore sizes can be adjusted by controlling the kinds of surfactants, ingredients or synthesis conditions employed during the production process.

Such material include, but are not limited to:

MCM-41 and MCM-48 (U.S. Pat. No. 5,057,296, U.S. Pat. No. 5,102,643). They are typically synthesized through a liquid crystal template pathway by using surfactants as templates. Based on the kind of surfactants or synthesis conditions their pore sizes can be adjusted in a range of 1.6 to 10 nm.

SBA-type material, such as SBA-1, SBA-C, SBA-3 (Science (1995) 268:1324) or SBA-15 (U.S. Pat. No. 6,592,764). Advantageously, SBA-15 can be readily prepared over a wide range of specific pore sizes (e.g. from 4.6-50 nm) and pore wall thicknesses at low temperature (35-80° C.) using a variety of commercially available, non-toxic and biodegradable amphiphilic block copolymers, including triblock polyoxyalkylenes.

An implantable device according to the present invention improves implant efficiency and prevents implant failure by biofilm-associated infections by supplying a suitable bioactive agent into the surrounding tissue through a slow or controlled release scheme. For instance, amorphous mesoporous silica can be synthesized by means of sol-gel processing inside the macroporous element of an implantable device with internal central cavity and/or porous reservoir and externally accessible injection port. The internal reservoir and/or central cavity itself can be filled with one or more slow release bioactive agents, yielding a long-lasting release (hours, days, weeks or even months) of the provided agent(s).

Thus, an implantable device according to the present invention may further comprise one or more bioactive agents or compositions preferably located in the macroporous element and/or the central cavity. Preferably, said bioactive agents or compositions comprise compounds or molecules capable of reducing, destroying, eradicating, inhibiting or preventing (fungal) biofilms or (fungal) biofilm formation on said implantable device.

Preferred bioactive compositions with anti-biofilm properties comprise one or more of the following compounds:

(i) antibiotics, preferably antimycotics, such as polyenes (e.g. amphotericin B, nystatin, natamycin); azoles (e.g. miconazole, fluconazole, itraconazole, voriconazole); allylamines (e.g. terbinafine); echinocandins (e.g. caspofungin); gentamycin, ofloxacin, ciprofloxacin; piperazine-1-carboxamidine derivatives (WO2010068296); antimicrobial peptides with antibiofilm activity; other antimycotic substances such as cetyltrimethylammonium bromide and the like;
(ii) agents that sensitize biofilms and/or persisters to existing antibiotics (antifungals), in particular an inhibitor of Efg1 function or expression, such as diclofenac or a derivative thereof, or a persister inhibitor such as N,N′-diethyldithio-carbamate or another superoxide dismutase inhibitor.

Accordingly, the present invention also relates to the combined use of such a sensitizing agent (e.g. Efg1 inhibitor) and at least one antimicrobial or antifungal agent for the treatment or prevention of fungal biofilm associated infections associated with the implantable device.

Preferably, one or more sensitizing agents, particularly an Efg1 antagonist, such as diclofenac, are released in a controlled way using the implant technology of the present invention, and the antibiotic/antimycotic is administered using a conventional route of administration, such as orally or intravenously. Alternatively, said sensitising agent may be coated on said implantable device. Advantageously, coating or incorporating only said sensitizing agent in the implant will not induce multi-drug resistance of the microorganisms following sustained exposure to these agents because such agents are not inhibitory themselves.

However, it is understood that various bioactive compositions and agents are suitable to be delivered locally by said fillable implantable device of the present invention.

Biological active agents suitable for being incorporated into the implant according to the present invention are preferably biological active agents which are capable of providing direct or indirect therapeutic, physiologic and/or pharmacologic effect in a human or animal organism. The biological active agent may include a drug, pro-drug, a targeting group or a drug comprising a targeting group.

Bioactive compositions for use with the aforementioned implant technology comprise bioactive agents, that include but are not limited to one or more biocides, anti-biofilm or biofilm sensitizing agents, antibiotic agents, antifungal agents, steroidal or non-steroidal anti-inflammatory agents, anti-inflammatory peptides (US20080311103), antiviral compounds, analgesics, painkillers, local anaesthetics, anticoagulants, antihypertensive substances, vitamins, or contrast media. Suitable compounds are well known to and are routinely selected by the skilled person.

In one aspect steroidal or non-steroidal anti-inflammatory agents may be included as bioactive agent in (onto) the present implantable device. Preferred steroidal anti-inflammatory agents comprise, but are not limited to, corticosteroids such as hydrocortisone, hydroxyltriamcinolon e, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene)acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, difluorosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof. Preferably, said steroidal anti-inflammatory is hydrocortisone. Preferred non-steroidal anti-inflammatory drugs (NSAIDs) include diclofenac, diflunisal, etodolac, fenoprofen, flurbiprofen, ibuprofen, indomethacin, ketoprofen, ketorolac, meclofenamate, mefenamic acid, meloxicam, nabumetone, naproxen, oxaprozin, piroxicam, salsalate, sulindac, tolmetin, celecoxib, rofecoxib.

In another aspect steroidal analgesics and/or local anesthetics may be used as bioactive agent in (onto) the present implantable device. Preferred steroidal analgesics include, but are not limited to aspirin, salicylic acid, diflunisal, morphine and its salts and the like. Preferred local anaesthetic agents include, but are not limited to procaine, benzocaine, lidocain, procain, bupivacaine, tetracain, xylocalne, mepivacaine and their salts and the like;

Alternatively, antiseptic substances, such as cetylpyridinium chloride, benzalkonium chloride, chlorhexidine and the like, may be included as a bioactive agent in (onto) the present implantable device.

Furthermore, antiprotozoals, such as chloramphenicol, sulfamethoxazole (and the like), may be included as a bioactive agent in (onto) the present implantable device.

Anticoagulants, e.g. heparin and its salts, such as calcium and sodium heparin, bishydroxycoumarin and the like, may be included as a bioactive agent in (onto) the present implantable device.

Antihypertensive agents, such as methyldopa, hydralazine, clonidine, chlorothiazide, timolol, propanolol, metroprolol, prazosin hydrochloride, furosemide and the like, may be included as a bioactive agent in (onto) the present implantable device.

Vitamins, such as vitamin B6, B12, C and the like, may be included as a bioactive agent in the present implantable device.

Other suitable biologically active agents include enzyme inhibitors, hormones, cytokines, growth factors, receptor ligands, antibodies, antigens, ion binding agents such as crown ethers and chelating compounds, substantial complementary nucleic acids, nucleic acid binding proteins including transcription factors, toxines etc. Examples of such active agents are, for example, cytokines such as erythropoietine (EPO), thrombopoietine (TPO), interleukines (including IL-1 to IL-17), insulin, insulin-like as growth factors (including IGF-1 and IGF-2), epidermal growth factor (EGF), transforming growth factors (including TGF-alpha and TGF-beta), human growth hormone, transferring, low density lipoproteins, high density lipoproteins, leptine, vascular endothelial growth factor (VEGF), fibroblast growth factor (FGF), PDGF, ciliary neurotrophic factor, prolactine, adrenocorticotropic hormone (ACTH), calcitonin, human chorionic gonadotropin, cortisol, estradiol, follicle stimulating hormone (FSH), thyroid-stimulating hormone (TSH), leutinizing hormone (LH), progesterone, testosterone, toxines including ricine and further active agents such as those included in Physician's Desk Reference, 58th Edition, Medical Economics Data Production Company, Montvale, N.J., 2004 and the Merck Index, 13th Edition (particularly pages Ther-1 to Ther-29).

The biologically active agent may preferably be selected from agents referring to angiogenesis, such as e.g. endostatin, angiostatin, interferones, platelet factor 4 (PF4), thrombospondin, transforming growth factor beta, tissue inhibitors of the metalloproteinases-1, -2 and -3 (TIMP-1, -2 and -3), TNP-470, marimastat, neovastat, BMS-275291, COL-3, AG3340, thalidomide, squalamine, combrestastatin, SU5416, SU6668, IFN-[alpha], EMD121974, CAI, IL-12 and IM862 etc., and further including combinations and/or derivatives of any of the foregoing.

The biological active agent may be selected from the group of drugs for the therapy of oncological diseases and cellular or tissue alterations.

The biologically active agent may be selected from the group of anti-viral, antibiotics and/or anti-infective drugs such as, in particular, β-lactam antibiotics, e.g., β-lactamase-sensitive penicillins such as benzyl penicillins (penicillin G), phenoxymethylpenicillin (penicillin V); β-lactamase-resistent penicillins such as aminopenicillins, e.g., amoxicillin, ampicillin, bacampicillin; acylaminopenicillins such as mezlocillin, piperacillin; carboxypenicillins, cephalosporins such as cefazoline, cefuroxim, cefoxitin, cefotiam, cefaclor, cefadroxil, cefalexin, loracarbef, cefixim, cefuroximaxetil, ceftibuten, cefpodoximproxetil, cefpodoximproxetil; aztreonam, ertapenem, meropenem; β-lactamase inhibitors such as sulbactam, sultamicillintosylate; tetracyclines such as doxycycline, minocycline, tetracycline, chlorotetracycline, oxytetracycline; aminoglycosides such as gentamicin, neomycin, streptomycin, tobramycin, amikacin, netilmicin, paromomycin, framycetin, spectinomycin; macrolide antibiotics such as azithromycin, clarithromycin, erythromycin, roxithromycin, spiramycin, josamycin; lincosamides such as clindamycin, lincomycin; gyrase inhibitors such as fluoroquinolones, e.g., ciprofloxacin, ofloxacin, moxifloxacin, norfloxacin, gatifloxacin, enoxacin, fleroxacin, levofloxacin; quinolones such as pipemidic acid; sulfonamides, trimethoprim, sulfadiazine, sulfalene; glycopeptide antibiotics such as vancomycin, teicoplanin; polypeptide antibiotics such as polymyxins, e.g., colistin, polymyxin-b, nitroimidazole derivates, e.g., metronidazole, tinidazole; aminoquinolones such as chloroquin, mefloquin, hydroxychloroquin; biguanids such as proguanil; quinine alkaloids and diaminopyrimidines such as pyrimethamine; amphenicols such as chloramphenicol; rifabutin, dapson, fusidic acid, fosfomycin, nifuratel, telithromycin, fusafungin, fosfomycin, pentamidine diisethionate, rifampicin, taurolidin, atovaquon, linezolid; virus static such as aciclovir, ganciclovir, famciclovir, foscarnet, inosine-(dimepranol-4-acetamidobenzoate), valganciclovir, valaciclovir, cidofovir, brivudin; antiretroviral active ingredients (nucleoside analogue reverse-transcriptase inhibitors and derivatives) such as lamivudine, zalcitabine, didanosine, zidovudin, tenofovir, stavudin, abacavir; non-nucleoside analogue reverse-transcriptase inhibitors: amprenavir, indinavir, saquinavir, lopinavir, ritonavir, nelfinavir; amantadine, ribavirine, zanamivir, oseltamivir or lamivudine, as well as any combinations and mixtures thereof.

Biologically active agents may also include anti-migratory, anti-proliferative or immune-supressive, anti-inflammatory or re-endotheliating agents such as, e.g., everolimus, tacrolimus, sirolimus, mycofenolate-mofetil, rapamycin, paclitaxel, actinomycine D, angiopeptin, batimastate, estradiol, statines and others, their derivatives and analogues.

When the implantable device of the present invention is introduced at least in part in bone, suitable biologically active agents include, but are not limited to such compounds that are used to treat bone tissue and in particular to enhance bone growth (e.g. bisphosphonate (e.g., risedronate, pamidronate, ibandronate, zoledronic acid, clodronic acid, etidronic acid, alendronic acid, tiludronic acid). Also, many growth factors from the TGF-β superfamily are relevant for a wide range of medical treatment methods and applications which in particular concern wound healing and tissue reconstruction. For a review of members of the TGF-β superfamily cf. e.g.: Roberts, A. B. & Sporn, M. B. Handbook of Experimental Pharmacology 95 (1990) 419-472; Kingsley, D. M., Genes & Development 8 (1994) 133-146 and the literature cited therein. The members include the TGF-β proteins such as TGF-β1, TGF-β2, TGF-β3, TGF-β4 and TGF-β5, The members of the BMP (bone morphogenetic protein) family form a further subfamily which include the proteins BMP-2 (BMP-2a), BMP-3, BMP-3b, BMP-4 (BMP-2b), BMP-5, BMP-6, BMP-7 (OP-1), BMP-8 (OP-2), BMP-9, BMP-10, BMP-11, BMP-12 and BMP-13. A further subgroup is the GDF (growth differentiation factor) family which include GDF-1, GDF-3, GDF-9, GDF-10, GDF-11 as well as GDF-5, GDF-6 and GDF-7 which are particularly interesting for cartilage-induction and/or bone-induction. It is known that many members especially from the subfamilies of the TGF-β, BMP and GDF families have a cartilage-inducing and/or bone-inducing potential and members of the activin family can also influence bone formation at least in combination with other TGF-β superfamily members. Since some of the individual proteins act at different sites during the course of bone induction, it can be assumed that a combination of various such proteins would be advantageous for the efficiency of cartilage and bone induction. Such protein mixtures are also encompassed by this invention.

A preferred aspect relates to an implantable device whereby the implant site or implant zone is in the form of a screw adapted to be screwed into a tissue, preferably a bone tissue (FIG. 12). This is particularly suitable for use as a dental implant. The solid or dense element of the dental implant device provides the structural basis for the attachment of a dental crown. The porous element allows fixation of the implant in the jaw bone by bone ingrowth. At any time after implantation the injection port can be accessed by removal of the dental crown, allowing administration of one or more biologically active agents into the implant. It is understood that the screw form is an example and that such implant can have different shapes.

Another preferred aspect relates to an implantable device whereby said implantable device is a bone implant.

The implantable device of the present invention may also be an abutment which provides the attachment for an external prosthesis, such as a prosthetic leg, finger or arm. As such an abutment permanently penetrates the skin, the tissue around the implant is sensitive to infection. With an injection port incorporated in the external part of the abutment localized administration of anti-inflammatory drugs or compounds and/or sensitizing agents with antibiofilm properties is rendered possible.

The implantable device of the present invention may also be a temporary support for a fixation cage. As with an abutment implant the skin is penetrated by the support, rendering it susceptible to infection. A porous section near the skin surface would allow administration of anti-inflammatory and antibiotic drugs in the case of complications.

The implantable device may also be an experimental device designed for in vivo testing or evaluation of new implant materials and/or controlled release systems and/or an experimental device designed for in vivo testing or evaluation of drugs such as antibiotics, biofilm inhibiting or sensitizing agents and anti-inflammatory agents and/or for in vivo testing or evaluation of functionalized implant surfaces.

The preferred device of the present invention, consisting of a macroporous titanium based structure filled with a mesoporous or microporous material, such as a metal oxide, preferably a silica, acting as a diffusion barrier of known porous properties, and with an (optional) incorporated central cavity accessible through an injection port, may also be used as an in vitro test kit.

Such a device may be placed in a micro well plate or similar test environment. This test setup enables to measure the amount of a biologically active compound, as described above, that is released through the diffusion barrier as a function of time.

In another experimental scheme a biofilm of known consistency can be grown on the device prior to antibiotic administration via the injection port. This allows testing the efficiency of the antibiotic (antifungal, and possibly sensitizing agent, such as Efg1 inhibitor) against biofilms. Using a similar experimental scheme combined with the administration of a biofilm sensitising compound onto the device or in the surrounding medium can be similarly used to test the efficiency of biofilm sensitizing agent and antibiotic combinations.

The device of the present invention can be used as in vitro test kit for the evaluation of controlled release methods. In this case a similar setup is used but the implant macroporous element is at least partially filled with a substance or medium enabling controlled release. The internal cavity is subsequently used to administer and test the rate of drug release via the previously administered means of controlled release or the efficiency of said drug administered using a controlled release.

The device of the present invention, comprising a macroporous titanium structure filled with a mesoporous or microporous material acting as a diffusion barrier of known porous properties, and with an (optionally) incorporated central cavity accessible through an injection port, is used as in vivo test kit for the evaluation of bioactive compounds such as antibiotics, antibiotic and biofilm sensitising compound combination or bone growth promoters. Either infection prevention by the released compounds or combinations of released and external compounds can be tested subcutaneously or interosseously. Similarly, a biofilm can be grown on the device prior to implantation allowing to test infection treatment by the controlled release, with or without externally present compound, or antibiotic.

The device of the present invention can be used as in vivo test kit for the evaluation of controlled release methods using the methods described in the previous embodiment. Prior to testing, the macroporous porosity of the porous element is charged with a controlled release medium. The device can then be used in any experimental procedure to test the efficiency of this controlled release method.

The implantable device can be implanted subcutaneously. The device then serves as a means for controlled administration of one or more biologically active agents, as listed above.

### EXAMPLES

### Example 1

### Efg1 Protects C. Albicans Biofilms Against Miconazole

Materials, yeast strains, plasmids and growth media. C. albicans homozygous deletion mutants used in this study are Δcdc35, Δras1, Δpde1, Δpde2 (Davis-Hanna et al. (2008) Mol. Microbiol. 67:47-62), Δefg1 (HLC52), the EFG1 reïntegrant Δefg1(EFG1) (HLC74) and the corresponding wild type CAF2. Growth medium used was YPD (1% yeast extract, 2% peptone, 2% glucose).

Biofilm activity assay. The activity of miconazole and fluconazole against 16 h-old C. albicans biofilms (10⁷ cells/well) was assessed using the crystal violet quantification method. The biofilm-eradicating capacity of a compound was determined as the minimal concentration resulting in 50% eradication of the biofilm (BEC50).

Quantitative analysis of intracellular accumulation of miconazole in C. albicans biofilm cells. Sixteen h-old C. albicans biofilms (10⁷ cells/well) were washed with PBS (pH 7.4) and incubated with 250 or 500 μg/ml miconazole in PBS for 4 h at 37° C. After incubation, treated biofilms were resuspended and pooled per 5 wells. After centrifugation, samples were taken for cell counting using a Thoma counting chamber, whereafter the pellet was resuspended in 300 μl 70% acetonitrile/30% PBS. Miconazole concentration in the cell lysates was determined using HPLC and normalized to the number of cells in the pellet. Statistical analysis was performed using unpaired student t test; differences were considered significant if p<0.05.

Expression analysis of EFG1 in biofilm cells. Cells of 16 h-old biofilms were collected and washed with physiological saline. Cell disruption, RNA purification, DNase treatment and RT-PCR were performed as described previously. After development of a forward (CTGCTTCGGCTCCTCCACCT; SEQ. ID. NO. 3) and reverse (CCTGCACCAGAAGCACCAGACA; SEQ. ID. NO. 4) primer for EFG1 and testing their specificity, real-time PCR was performed using a Mesa Green qPCR kit 71 (Eurogentec). The expression level of EFG1 in both conditions was normalized using two reference genes (ACT and RPP2B). Experiments were repeated three times.

Results

### ΔEfg1 Biofilms are Hypersensitive to Miconazole.

Miconazole sensitivity was analyzed of biofilms of C. albicans homozygous null mutants affected in genes involved in the cyclic AMP signaling pathway, namely Δcdc35, Δras1, Δpde1, Δpde2 and Δefg1. To this end, the inventors incubated 16 h-old C. albicans biofilms with various concentrations of miconazole for 24 h and determined the remaining biomass using the crystal violet quantification method.

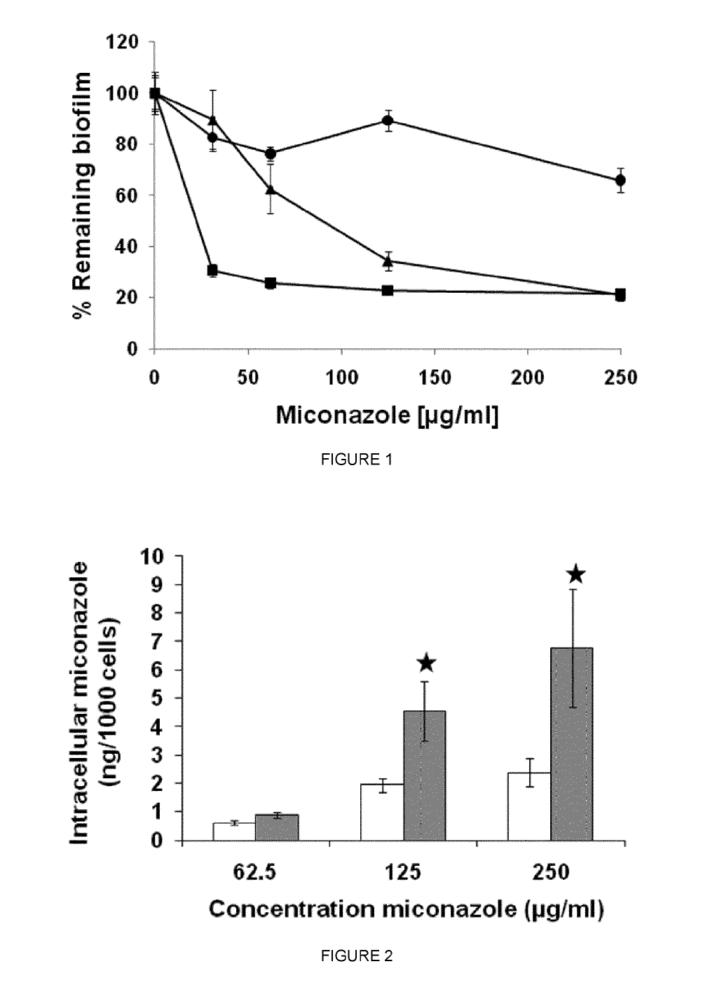

Only Δefg1 biofilms showed increased sensitivity to miconazole (BEC50=25 μg/ml) as compared to biofilms of the corresponding isogenic WT strain CAF2 (BEC50>250 μg/ml) and the reintegrant Δefg1(EFG1) (BEC50=100 μg/ml) (FIG. 1). In contrast to WT or Δefg1(EFG1) biofilms, biofilms of Δefg1 were sensitive to miconazole upon incubation with a low miconazole dose (31 μg/ml).

Following incubation with moderate miconazole doses (62-125 μg/ml), biofilms of the reïntegrant Δefg1(EFG1) were characterized by increased miconazole tolerance as compared to biofilms of Δefg1, but also by increased miconazole sensitivity as compared to WT biofilms, pointing to an intermediate miconazole sensitivity phenotype of sessile Δefg1(EFG1) cells.

In line with this observation, the inventors found the EFG1 expression levels in Δefg1(EFG1) biofilms to be 18 times lower than those in WT biofilms, explaining the observed intermediate miconazole phenotype of Δefg1(EFG1) biofilms. Following incubation with a high miconazole dose (250 μg/ml), biofilms of Δefg1(EFG1) were as sensitive to miconazole as Δefg1 biofilms, whereas WT biofilms were resistant to this miconazole dose. [0206]

Intracellular Miconazole Levels are Increased in ΔEfg1 Biofilms.

The intracellular accumulation of miconazole in Δefg1 and WT biofilms upon miconazole treatment was determined to investigate whether the increased miconazole sensitivity of Δefg1 biofilms is due to an increased uptake or decreased efflux of miconazole. To this end, 16 h-old Δefg1 and WT C. albicans biofilms were treated with different concentrations of miconazole for 4 h and the concentration of miconazole in the biofilm cells via HPLC analysis was determined. Treatment of Δefg1 or WT biofilms with 62-250 μg/ml miconazole resulted in significantly increased intracellular miconazole levels in Δefg1 biofilm cells as compared to WT biofilm cells (FIG. 2). These data indicate that the miconazole sensitivity of Δefg1 biofilms is probably due to increased intracellular miconazole accumulation in Δefg1 biofilm cells as compared to WT biofilm cells.

The above results aided in unraveling part of the molecular basis responsible for the occurrence of tolerance of C. albicans biofilms against miconazole. Using an in vitro biofilm model, the inventors found that biofilms of the Δefg1 C. albicans mutant exhibited increased miconazole sensitivity as compared to WT biofilms. Concomitantly, a significant increase in intracellular miconazole levels in Δefg1 biofilm cells was observed as compared to WT cells following miconazole treatment, explaining the increased miconazole sensitivity of Δefg1 biofilm cells. The data point to the presence of an import- or efflux-dependent miconazole tolerance mechanism in Candida biofilms involving Efg1.

### Example 2

### Diclofenac Increases the Sensitivity of C. Albicans WT Biofilms to Miconazole

Wild-Type C. albicans cultures were treated with 500 μg/ml diclofenac during their biofilm growth phase and the miconazole-sensitivity of the resulting biofilms was assessed.

To this end, cultures of the WT CAF2-1 were grown overnight (2×10⁸ cells/ml) in YPD (1% yeast extract, 2% peptone, 2% glucose) and washed 3 times in PBS (pH 7.4). After dilution (OD600=0.5) in SC (1% CSM, complete amino acid supplement mixture, 1% YNB, yeast nitrogen base; 2% glucose), the cultures were resuspended (100 μl/well) in a 96well microtiter plate. After 1 h of adhesion, the adherent WT cells were incubated with or without 500 μg/ml diclofenac and allowed biofilm formation for 16 h. Next, the resulting biofilms were washed with PBS and treated with various concentrations of miconazole for 24 h in the absence or presence of diclofenac. After washing, the remaining biomass was determined using the crystal violet quantification method.

The biofilm-eradicating capacity of a compound was determined as the minimal concentration resulting in 50% eradication of the biofilm (BEC50).

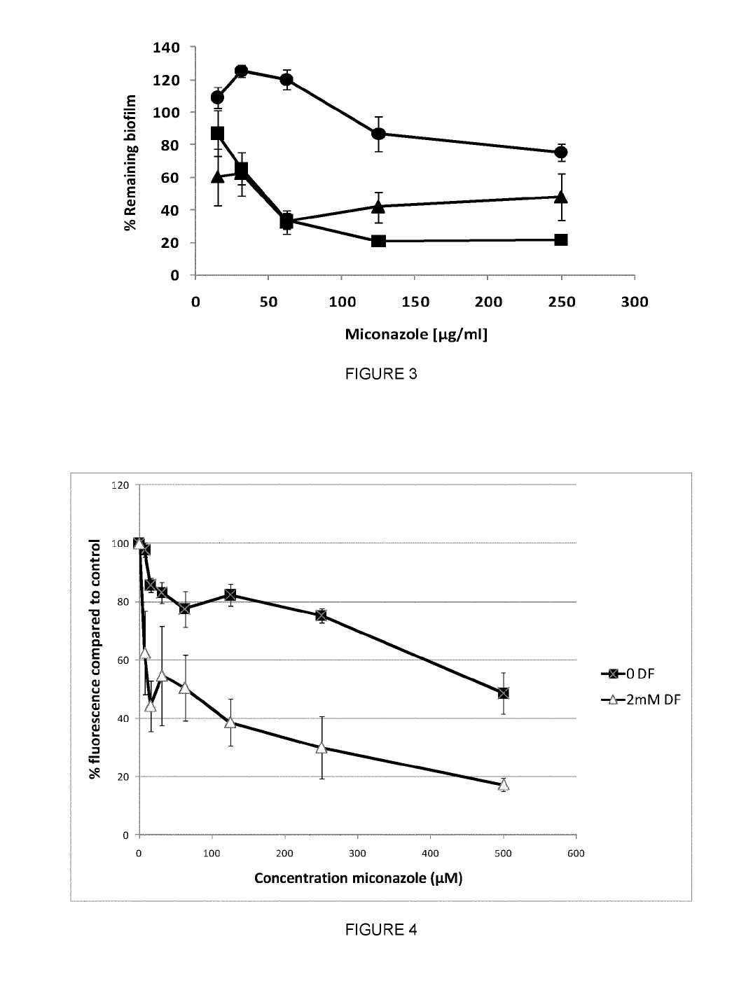

WT biofilms treated with 500 μg/ml diclofenac showed increased sensitivity to miconazole (BEC50=46 μg/ml) as compared to untreated WT biofilms (BEC50>250 μg/ml) (FIG. 3).

Treatment of biofilms with 500 μg/ml diclofenac in the absence of miconazole resulted in about 50% remaining C. albicans biofilms when compared to untreated WT biofilms, indicating that diclofenac itself has only a moderate biofilm-inhibitory effect in vitro and on non-established biofilms.

These data were further confirmed using an alternative biofilm quantification method, based on live/dead staining via CellTiter-Blue (Promega). To this end, cultures of the WT CAF2-1 were grown overnight (2*10⁸ cells/ml) in YPD (1% yeast extract, 2% peptone, 2% glucose) and washed 3 times in PBS (pH 7.4). After dilution (OD600=0.5) in SC (1% CSM, complete amino acid supplement mixture, 1% YNB, yeast nitrogen base; 2% glucose), the cultures were resuspended (100 μl/well) in a 96well microtiter plate. After 1 h of adhesion, the adherent WT cells were incubated with or without 2 mM (about 500 μg/ml) diclofenac (in DMSO, resulting in final concentration of 2.4% DMSO) and allowed biofilm growth for 16 h in SC. Next, the resulting biofilms were washed with PBS and treated with various concentrations of miconazole (0-500 μM) for 24 h in the absence or presence of diclofenac. After washing, the remaining living cells using the CellTiter-Blue quantification method were determined. WT biofilms treated with 2 mM diclofenac showed increased sensitivity to miconazole as compared to untreated WT biofilms (FIG. 4).

### Example 3

### Intracellular Miconazole Levels are Increased in Diclofenac-Treated WT Biofilms

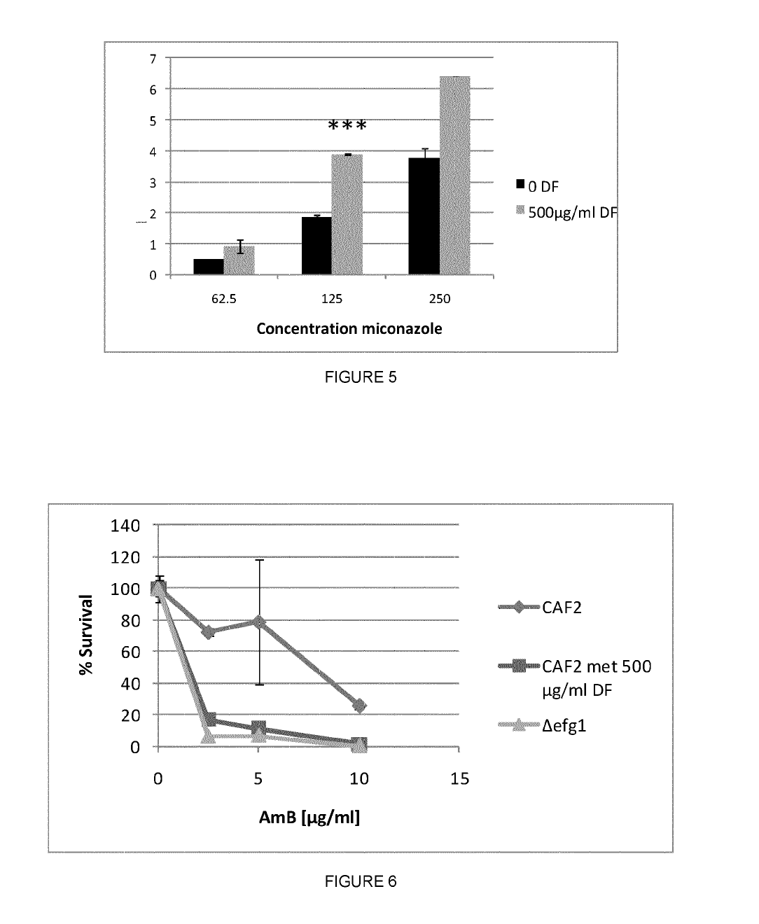

The intracellular accumulation of miconazole in WT biofilms grown in the prescence or absence of 500 μg/ml diclofenac upon miconazole treatment was determined to investigate whether the increased miconazole sensitivity of diclofenac-treated WT biofilms is due to an increased uptake or decreased efflux of miconazole. To this end, biofilms were grown as described above, and subsequently treated with various concentrations of miconazole in the absence or presence of diclofenac. Samples were taken for cell number determination using a Thoma counting chamber, whereafter the isolated biofilm cells were resuspended in 300 μl 70% acetonitrile/30% PBS. Miconazole concentration in the cell lysates was determined using a HPLC setup as described previously (Bink et al. (2010) FEMS Yeast Research 10, 812-818.) and normalized to the number of cells in the pellet. Treatment of WT biofilms with 62-250 μg/ml miconazole and 500 μg/ml diclofenac resulted in significantly increased intracellular miconazole levels in diclofenac-treated WT biofilm cells as compared to untreated WT biofilm cells (FIG. 5). These data indicate that the increased miconazole sensitivity of diclofenac-treated WT biofilms is probably due to increased intracellular miconazole accumulation in these biofilm cells as compared to WT biofilm cells grown in the absence of diclofenac. These results are in line with the increased miconazole sensitivity of Δefg1 biofilm cells due to the increased accumulation of intracellular miconazole compared to WT biofilm cells.

This was further investigated by evaluating the membrane-disruptive activity of diclofenac on in vitro grown C. albicans biofilm cells by measuring the fluorescence enhancement of propidium iodide (PI). To this end, C. albicans biofilms were grown in RPMI 1640 (pH 7.0) in the presence or absence of diclofenac (ranging from 0.25-4 mM) for 24 h. Next, the biofilm cells were incubated with 3% PI for 20 minutes at room temperature in the dark. Membrane permeability was quantified by measurement of PI fluorescence (excitation at 535 nm, emission at 617 nm). The results clearly show that C. albicans cells treated with 2 mM diclofenac during the adhesion and growth phase of biofilm formation resulted in significantly increased membrane permeability of C. albicans biofilm cells compared to untreated C. albicans cells using propidium iodide staining (***p<0.0001).

### Example 4

### Diclofenac Increases the Sensitivity of C. Albicans WT Biofilms to Amphotericin B

Using the same setup as in Example 2, WT biofilms grown in the presence or absence of diclofenac (500 μg/ml) or Δefg1 biofilms were incubated with various concentrations of Amphotericin B (AmB) (2.5-10 μg/ml) for 24 h, both in the presence or absence of diclofenac. After washing, the remaining living cells within the biofilm were determined by plating on YPD and determination of colony forming units (CFUs).

WT biofilms treated with 500 μg/ml diclofenac showed increased sensitivity to Amphotericin B as compared to diclofenac-untreated WT biofilms (FIG. 6): % survival of WT biofilms grown in the presence of diclofenac and treated with various AmB concentrations dropped about 5-fold as compared to diclofenac-untreated biofilms.

In this example, biofilms grown in the presence of 500 μg/ml diclofenac alone contained about 50% less biofilm cells as compared to untreated WT biofilms, indicating that diclofenac itself has only a moderate biofilm-inhibitory effect in vitro and on not yet established biofilms.

### Example 5

### Diclofenac Increases the Sensitivity of C. Albicans WT Biofilms to Caspofungin (In Vitro)

Using the same setup as above, WT biofilms grown in the presence or absence of diclofenac (0.5-3 mM or 148-888 μg/mL) were incubated with various concentrations of Caspofungin (0-150 μM) for 24 h, both in the presence or absence of diclofenac. After washing, the remaining living cells within the biofilm was determined by CellTiter-Blue quantification. The MIC50 of caspofungin, diclofenac and the combination of both drugs against pretreated or mature C. albicans biofilms was quantified by XTT reduction assay (Ramage et al.).

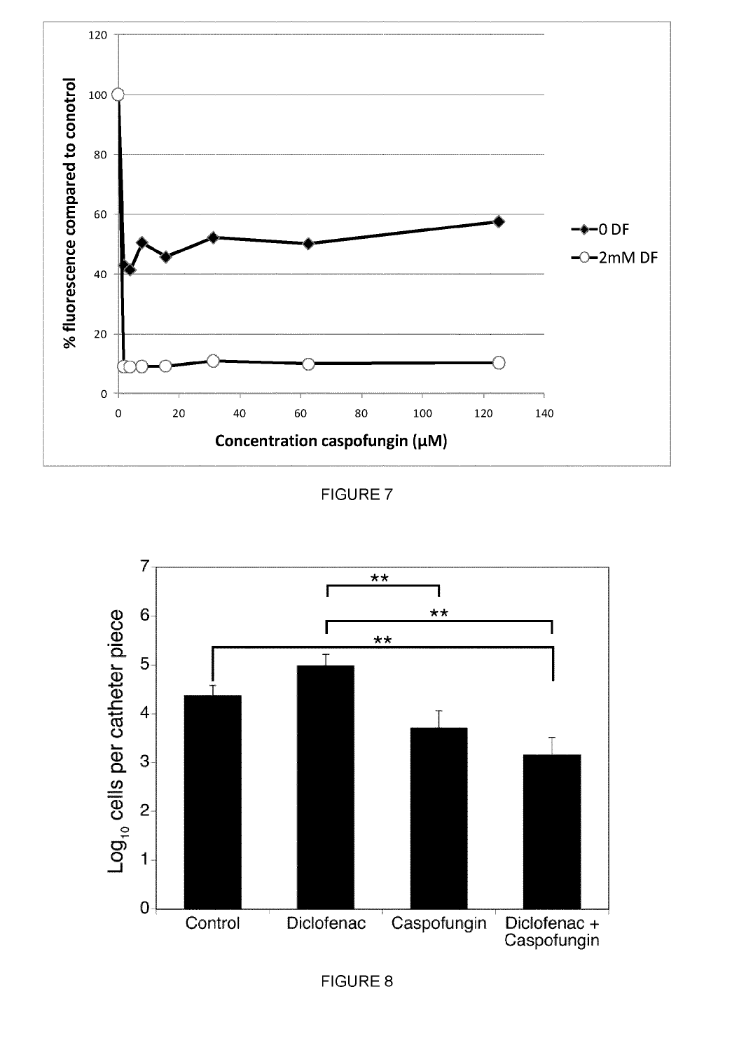

WT biofilms treated with 0.5-3 mM diclofenac (during adhesion and throughout the biofilm formation) showed increased sensitivity to caspofungin as compared to diclofenac-untreated WT biofilms (FIG. 7): % survival of WT biofilms grown in the presence of diclofenac and treated with various caspofungin concentrations dropped about 5-fold as compared to diclofenac-untreated biofilms.

Similar results were obtained by determination of the MIC50 values. The susceptibility testing and determination of minimal inhibitory concentrations (MIC) of diclofenac and caspofungin, and combinations thereof that inhibit fungal growth by 50% (MIC50) were performed according the NCCLS M27-A3 protocol (2008).

The MIC50 of caspofungin and/or diclofenac against planktonic cultures of C. albicans CAF2-1 in RPMI 1640 medium showed no increased activity of caspofungin in combination with diclofenac against planktonic cultures. MIC50 of caspofungin against planktonic cultures was <0.05 μM, whereas MIC50 of the combination of caspofungin and 2 mM diclofenac in this setup was 0.23 μM.

Moreover, the inventors observed no inhibitory activity of diclofenac up to 2 mM against planktonic cells nor an effect of diclofenac on the morphology of the planktonic cells, as analysed by fluorescence microscopy.

The MIC50 of caspofungin against biofilms of C. albicans CAF2-1 was 6 μM, which was at least 100 times higher than the MIC50 of caspofungin against planktonic cultures (MIC50<0.05 μM), documenting the strongly increased resistance of C. albicans biofilms against caspofungin.

Diclofenac alone had a moderate antibiofilm effect (MIC50=2 mM). However, the morphology of biofilm cells pretreated with diclofenac alone (ranging from 0.5-3 mM), was not altered.

The MIC50 of caspofungin in combination with 2 mM diclofenac-pretreatment against C. albicans biofilms was <0.75 μM, whereas coincubation of mature biofilms with caspofungin and 2 mM diclofenac resulted in MIC50 of 1.5 μM.

FICI values for the diclofenac pretreatment/caspofungin or diclofenac coincubation/caspofungin combinations against C. albicans biofilms were 0.125 and 0.25, respectively. These FICI values, both <0.5, point to the synergistic interaction between caspofungin and diclofenac on biofilms (Moody 1991), or stated differently, diclofenac makes the biofilm cells more susceptible to the antifungal agent.

All these data indicate that the caspofungin potentiation by diclofenac is biofilm-specific and most pronounced upon diclofenac pretreatment. Similar results were obtained in the case of biofilms of C. albicans clinic isolates or caspofungin resistant strains.

In an alternative setup, in vitro biofilm drug susceptibility assays were performed using serum-coated 1 cm polyurethane catheter pieces (Arrow International Reading) as described previously. Biofilms were grown in RPMI 1640 (pH 7.0) and Candida cells were allowed to adhere to the substrate for 90 min.

Diclofenac (2 mM) was administered during the period of adhesion of C. albicans cells on the catheters and throughout the period of biofilm formation. Catheter fragments containing diclofenac-pretreated or untreated mature biofilms were washed and subsequently incubated with caspofungin (125 μM) or water in the presence or absence of diclofenac (2 mM) for 24 h, respectively. Biofilms were washed and quantified by determination of colony forming units. Untreated biofilms were considered as controls. Statistical analysis was performed using unpaired t-test; differences were considered significant if p<0.05. Data are the mean and SEM of five independent experiments using at least two catheters per test group. The numbers of sessile C. albicans cells recovered from the treated catheters are given in FIG. 8.

Treatment of C. albicans biofilms grown on catheters with 125 μM caspofungin did not result in a statistically significant reduction of the number of biofilm cells compared to the control treatment. Likewise, the inventors found no significant difference in the number of biofilm cells that were retrieved from catheters upon diclofenac pretreatment alone compared to untreated catheters.

Treatment of C. albicans-inoculated catheters with 2 mM diclofenac during biofilm growth and subsequent treatment with 125 μM caspofungin resulted in an at least 12-fold significant reduction of the number of in vitro biofilm cells compared to the untreated biofilms, again demonstrating to a potentiation of the in vitro antibiofilm activity of caspofungin by diclofenac.

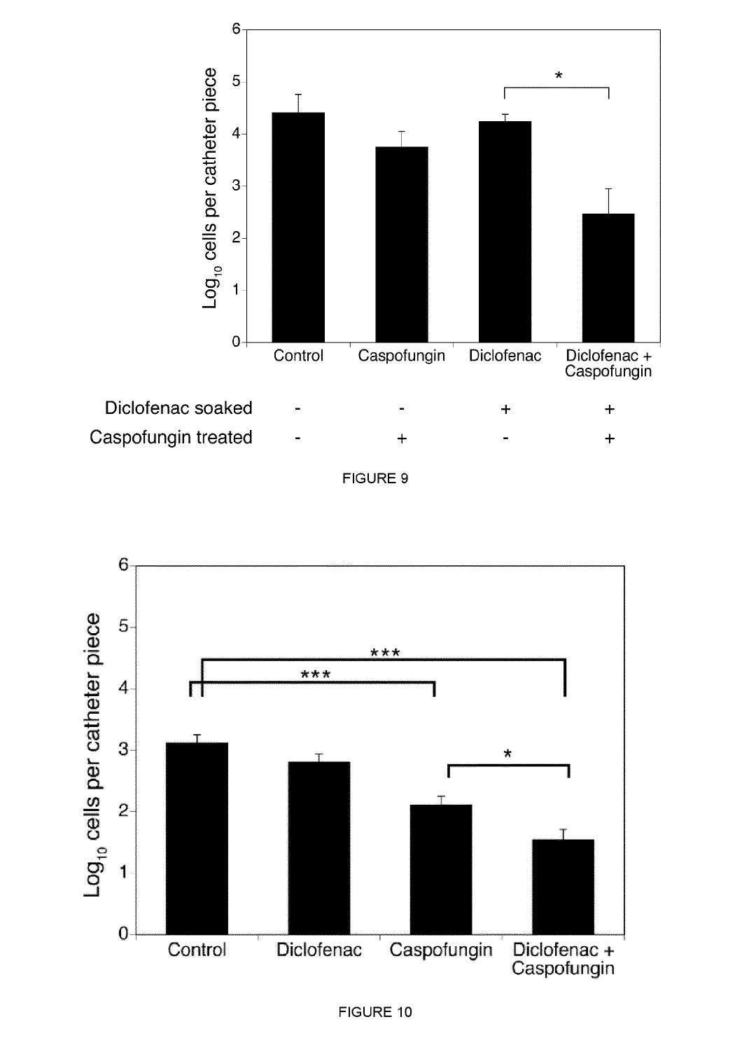

Similar results were obtained when assessing the antibiofilm effect of caspofungin against mature biofilms, which were formed on diclofenac-soaked catheter fragments versus those formed on unsoaked catheter fragments. A caspofungin dose (125 μM) that did not display significant activity against mature biofilms grown on unsoaked catheter fragments, could significantly reduce the number of biofilm cells of biofilms grown on diclofenac-soaked catheters by at least 17-fold as compared to control treatment (*p<0.05) (FIG. 9).

This implies that therapeutically effective diclofenac concentrations can be achieved in coatings or controlled release formulas on e.g. medical devices. Indeed, the data at least point to the feasibility of achieving a sufficiently high diclofenac concentration on cathethers after soaking them in a diclofenac solution in order to affect biofilm susceptibility for caspofungin.

### Example 6

### Diclofenac Potentiates the In Vivo Activity of Caspofungin Against C. Albicans Biofilms

Materials & methods. Animals were maintained in accordance with the appropriate animal care guidelines and animal experiments were approved by the ethical committee of the K. U. Leuven.

C. albicans CAF2 was grown routinely on YPD (1% yeast extract, 2% peptone, 2% glucose) agar plates at 37° C. Stock solutions of caspofungin (Cancidas, Merck) and diclofenac were prepared in sterile water. Phosphate buffered saline (PBS) was prepared by combining 8 g/l NaCl, 0.2 g/l KCl, 1.44 g/l Na₂HPO₄ and 0.24 g/l KH₂PO₄ (pH 7.4). RPMI 1640 medium with L-glutamine, without bicarbonate (pH 7.0) was purchased from Sigma.

A rat subcutaneous catheter infection model was used for in vivo biofilm susceptibility studies, in which 6 rats were treated with sterile saline (control, 40 catheters), 6 rats with diclofenac (48 catheters), 5 rats with caspofungin (40 catheters) and 6 rats with a combination of diclofenac and caspofungin (45 catheters).

Diclofenac and caspofungin solutions for intravenous injection were prepared in sterile physiological solution. The administration of diclofenac (3 mg/kg) started immediately after the catheter implant and continued daily up to 9 days. The treatment with caspofungin (3 mg/kg) was initiated after 48 h of in vivo biofilm development and maintained by daily injections for 7 days. Sterile saline was administered to the control group. Further analysis and quantification of the number of cells per individual biofilm was performed as previously described. Statistical analysis was performed using unpaired t-test; differences were considered significant if p<0.05. Data are the means and SEM of 2 independent experiments. Six independent catheters retrieved from each test group were used for scanning electron microscopy (SEM) analyses. Prior to the SEM analysis, each catheter fragment was cut longitudinally through the lumen and subsequently prepared for microscopy as previously described.

To assess the increased sensitivity of diclofenac-pretreated C. albicans biofilms to caspofungin under in vivo conditions, a subcutaneous biofilm model system in rats was used.

The numbers of sessile C. albicans cells recovered from the implanted catheters after various treatments are represented in FIG. 10. There was no significant difference in the number of biofilm cells on catheters retrieved from rats receiving no drugs (control treatment) or receiving 48 h diclofenac-pretreatment and further diclofenac-treatment (3 mg/kg) up to 7 days, indicating that diclofenac treatment alone does not affect in vivo growing biofilms.

Caspofungin treatment alone, i.e. administered at 3 mg/kg by daily injections for 7 days without diclofenac-pretreatment of the rats, resulted in a more than 10-fold significant reduction in the number of surviving C. albicans biofilm cells on the implanted catheters compared to control treatment (***p<0.001).

However, daily caspofungin treatment for 7 days in combination with 48 h diclofenac-pretreatment and further diclofenac-treatment up to 7 days resulted in a more than 15-fold significant reduction in biofilm cell numbers compared to control treatment and in a more than 5-fold significant reduction in cell numbers compared to caspofungin treatment alone (***p<0.001 and *p<0.05, respectively).

These observations are reflected in the number of sterile catheter fragments retrieved from the different treatment groups: three catheters (7.5%) retrieved from the animals treated with caspofungin alone were sterile, whereas thirteen sterile catheters (29%) were retrieved from the animals treated with both caspofungin and diclofenac, pointing to a much more pronounced effect and/or to a more pronounced effect towards persisters.

C. albicans biofilm structure and morphology of various catheters per animal was also evaluated using scanning electron microscopy. Catheters retrieved from untreated and diclofenac-pretreated rats showed dense patches of typical C. albicans biofilm architecture alongside the catheter lumen, composed of a hyphal network and covered with a layer that can be considered as extracellular matrix. Catheters retrieved from rats treated with caspofungin without diclofenac-pretreatment showed C. albicans biofilm cells formed in minor clusters, still revealing biofilm structure similar to untreated biofilms with signs of extracellular polymeric material present on top. The scanning electron microscopy data demonstrated that diclofenac alone has no biofilm-inhibitory effect.

However, only scattered yeast cells and hyphae were observed after treatment of the animals with diclofenac and caspofungin together, confirming the data based on CFU determination.

The above examples demonstrate that treatment of C. albicans biofilms with the NSAID diclofenac phenocopies the effect of EFG1 deletion in C. albicans biofilms and sensitizes C. albicans biofilms to various antimycotics like miconazole, Amphotericin B and caspofungin: C. albicans biofilms grown in the presence of diclofenac showed increased sensitivity to these antifungals.

Also, the data reveal successful reduction of in vitro and in vivo C. albicans biofilms on a medical device, particularly catheters, by combination therapy of the anti-inflammatory drug diclofenac and the antimycotic caspofungin.

These results provide clear evidence that an Efg1 inhibitor, preferably diclofenac or a derivative thereof, is useful in combination therapy with antifungals like miconazole, Amphotericin B or caspofungin (or other conventional antifungal agents) to treat C. albicans-biofilm associated infections.

More specifically, the inventors have disclosed that diclofenac-coated, diclofenac-soaked or diclofenac-releasing medical devices, like implants and plastics, are ideally suited for the elimination of C. albicans biofilms in combination with conventional antifungal therapy. This is in contrast to diclofenac or the antifungals alone, which are not very effective against these biofilms.

### Example 7

### Synthesis of a Mesoporous or Microporous Material in a Macroporous Implant Element

1) A typical synthesis procedure for the SAPO-5 zeolite (zeolite type AFI) uses aluminum isopropoxide as an aluminum source, phosphoric acid as a phosphorus source, tripropylamine (TPA) as molecular template, and Aerosil200 (Degussa) as a silica source. An optimized synthesis gel has the following composition:

Al₂O₃:P₂O₅:TPA:H₂O:Ethanol:SiO₂—1:0.99:1.2:400:20:0.01.

A patterned macroporous implant piece is immersed in the synthesis gel, and both are put in a sonication bath for 15 min at 47 kHz. Excess synthesis gel is removed via a spin coating procedure, using 15000 rpm for 10 s. The crystallization is carried out under autogeneous pressure for 14 h-15 h at 180° C. Afterwards, the autoclaves are cooled in air, and the macroporous implant piece is thoroughly rinsed with doubly-distilled water. The macroporous implant piece is again sonicated to remove the non-attached crystals, and again thoroughly rinsed with doubly-distilled water.

2) Another method to growth zeolites into macropores has been described by Hiroko Shikata et al. (2000), Journal of Sol-Gel Science and Technology 19, 769-773. In this procedure, tetraethoxysilane (TEOS, Shin-Etsu Chemical Co.) is used as a as a silica source and tetrapropylammonium hydroxide (TPAOH, Tokyo Chemical Industry Co.) is used as an alkaline source as well as an organic template including the use of other materials tetrapropylammonium bromide (TPABr, Tokyo Chemical Industry Co.) and distilled water. A precursor solutions of TPA-silicalite-1 was prepared by mixing TEOS, 10% TPAOH aqueous solution, TPABr, and H₂O. The molar composition is

TPAOH:TPABr:TEOS:H₂O—3:7.5:25:3000.

This synthesis solution is consequently stirred at about 40° C. for varied periods of time, and then poured into a polytetrafluoroethylene vessel, into which a macroporous implant element is immersed. The vessel is sealed in a stainless autoclave and heated at about 100° C. for 72 h without agitation. Thereafter, the samples were dried at about 40° C. in air for more than 24 h.

3) Amorphous microporous silica materials can be coated onto and in part into the macroporous implant element. Said silica materials are prepared by combining (i) a silicon alkoxide source, (ii) a solvent, and (iii) an acid catalyst (HCl solution). The HCl-solution is added dropwise to a stirred solution of silicon alkoxide in solvent in a suitable container, for instance in a polytetrafluoroethylene vessel. Next, the macroporous implant is immersed in the mixture and stirring is continued for 24 h at room temperature. After removal of mixture impregnated implant from said mixture, it is put on the side of the solid material element and is heated at 40° C. under quiescent conditions in a furnace for 3 days. A stiff transparent gel coating is obtained. This is heated to 65° C. with a heating rate of 0.1° C./min. After 5 h at 65° C. the material was heated to the final temperature of 250° C. (at a heating rate of 0.1° C./min). After 5 h at 250° C. the coated implant device was cooled to ambient temperature.

### Example 8

### Controlled or Slow Release Properties of a Macroporous Element Comprising a Mesoporous Material Embedded in the Macroporous Element

### Synthesis & Characterisation

SiO₂ is deposited in the internal volume of sintered titanium discs according to the following procedure.

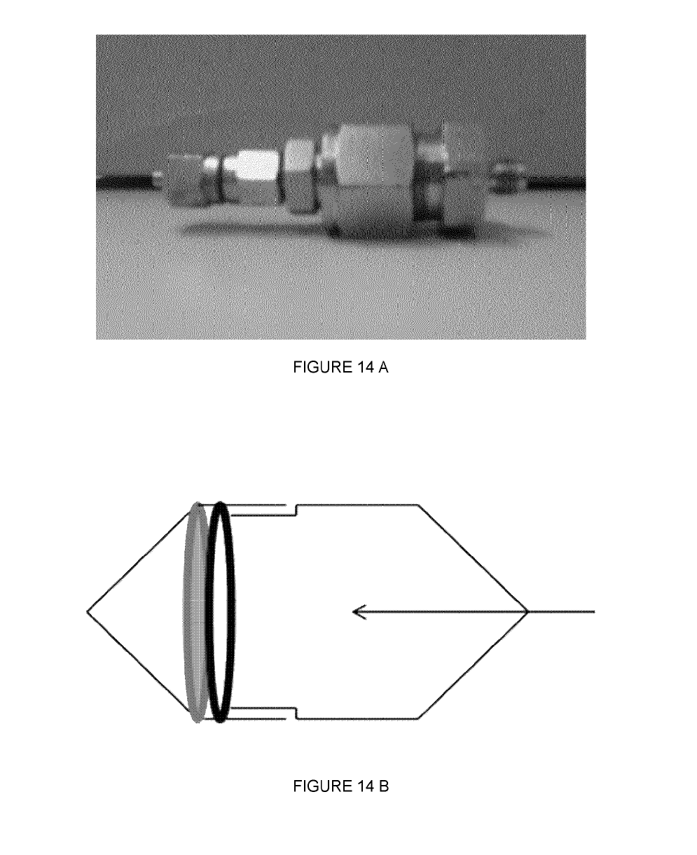

A titanium disc of dimensions (d×h: 12.74 mm×1.18 mm) is mounted in a Swagelok® valve body (SS-CHS2-1) and a viton o-ring is mounted on the Ti disc to ensure closure of the Swagelok® body. This is schematically shown in FIG. 14.
Synthesis solution A is prepared by mixing 6 ml of Ludox HS-40 and 12 ml HCl solution with pH 1.25 in a PP beaker.
Synthesis solution B is prepared by mixing 3 ml of Ludox HS-40 and 3 ml HCl solution of pH 1.25 in a PP beaker.
The respective solutions are mixed for 1 h and consequently loaded in a 10 ml syringe. This syringe is attached to the Swagelok® body containing the Ti disc, the synthesis solution is forced through the Ti disc until 2 ml could be recovered on the other side and the system is allowed to rest for 1 h.

After removal of the syringe, the body is opened and the remaining synthesis solution is removed. The disc is allowed to dry at ambient temperature and atmosphere overnight. Next, a 6 h calcination is performed at 225° C. with a heating ramp of 1° C./min. Careful positioning of the disc was performed so that both sides were open to the atmosphere.

The resulting Si-containing Ti discs were characterized by nitrogen adsorption performed on a Quantachrome Autosorb-1 apparatus. Prior to measurement, both samples A and B (Ti disc+SiO₂ deposition) were pretreated at 200° C. for 10 h under vacuum. The nitrogen adsorption/desorption isotherm of both calcined samples shows a hysteresis loop, indactive of the presence of mesopores. The pore size distribution is narrow with a mean pore diameter of approximately 8 nm for sample A and approximately 6 nm for sample B.

### Transport/Diffusion Restriction

The transport restriction imposed by the SiO₂-deposition was measured in an air flow setup. Using a pressure transducer one side of the Ti disc was exposed to a feed pressure of 3 bar. The flow was measured using a digital flow meter. A reference Ti disc allowed gas transport at 1800 ml/min. The sample A SiO₂-impregnated Ti disc only allowed 32 ml/min equal to a flow reduction of 51.5 times, while the sample B SiO₂-impregnated Ti disc only allowed 15.1 ml/min, equal to a flow reduction of 119.2 times.

A percolation test was performed using aqueous methylene blue solutions. Prior to this test the internal volume of the Ti discs is saturated with water using a high pressure pump. The feed side of the Swagelok® body is filled with 3 ml of methylene blue solution (50 mM) and sealed with a Swagelok® stopcock. The collector side is filled with approximately 0.3 ml of distilled water in such a way that no air bubbles are present and closed with a Swagelok® stopcock. During the percolation test the Swagelok® body is positioned such that the Methylene blue reservoir is resting on top of the Ti disc. During sampling the body is positioned upside down and the collector side is emptied with a syringe and needle. The methylene blue concentration at the collector side was quantified by UV-absorption relative to a calibration line. FIG. 11A and 11B show the methylene blue concentration at the collector side relative to the feed concentration and clearly shows the slow/controlled release characteristics of the samples.

1.-27. (canceled)

28. A medical implant device, comprising:
a solid structural element;
a first porous element, which is attached to or bonded with said solid element, said first porous element having a pore size in the range of 50 nm to 1000 μm; and
a second porous material, being an amorphous silica material, having a pore size in the range of 1 to 30 nm and embedded in said first porous element or surrounding said first porous element, wherein said first porous element is in ceramic materials, in metals or in metal alloys.

29. The medical implant device of claim 28 further comprising an injection port incorporated in said solid structural element, wherein said injection port allows delivery of a bioactive agent into the porous part of the medical implant device.

30. The medical implant device of claim 29 further comprising a cavity able to contain a fluid comprising a bioactive agent, wherein said cavity is connected with both the injection port and the porous element.

31. The medical implant device of claim 30, wherein the cavity is located in the solid element of the implant or extends into the first porous element and/or the second porous material.

32. The medical implant device of claim 28, wherein the first porous element is in titanium or in titanium derivatives.

33. The medical implant device of claim 28, wherein the first porous element has a pore size in the range of 200 nm to 1000 nm.

34. The medical implant device of claim 30, wherein the first porous element has a pore size in the range of 200 nm to 1000 nm.

35. The medical implant device of claim 28, wherein the second porous material has a pore size in the range of 2 nm to 25 nm.

36. The medical implant device of claim 30, wherein the second porous material has a pore size in the range of 2 nm to 25 nm.

37. The medical implant device of claim 28, comprising a surface of a medical device selected from the group consisting of catheters, implants, prostheses, stents, surgical plates, valves or pins, artificial joints, pacemakers, contacts lenses and bio-implants.

38. The medical implant device of claim 28, wherein the medical implant device is a dental implant.

39. The medical implant device of claim 30, wherein the bioactive agent comprises a pharmaceutical compound selected from the group consisting of antibiotics, antifungal agents, sensitizing agents, anti-inflammatory agents, analgesic agents and a mixture thereof.

40. The medical implant device of claim 30, wherein the implantable device comprises a pharmaceutical composition capable of reducing, inhibiting or preventing microbial biofilm formation.

41. The medical implant device according to claim 40 wherein the pharmaceutical composition comprises an anti-inflamatory agent and/or an Efg1 inhibitor.

42. The medical implant device according to claim 41 wherein the Efg1 inhibitor is diclofenac.

43. The medical implant device according to claim 39 wherein the pharmaceutical composition comprises an antifungal agent selected from the group consisting of hormones, cytokines, growth factors, antibodies, immune-suppressive, antineoplastic agents and combination thereof.

44. The medical implant device according to claim 42 wherein the pharmaceutical composition comprises an antifungal agent selected from the group consisting of hormones, cytokines, growth factors, antibodies, immune-suppressive, antineoplastic agents and combination thereof.

45. The medical implant device according to claim 39 wherein the pharmaceutical composition comprises an antifungal agent selected from the group consisting of caspofungin, miconazole and amphotericin.

46. The medical implant device according to claim 42 wherein the pharmaceutical composition comprises an antifungal agent selected from the group consisting of caspofungin, miconazole and amphotericin.

47. The medical implant device according to claim 39 wherein said pharmaceutical composition further comprises a second biologically active agent selected from the group consisting of hormones, cytokines, growth factors, antibodies, immune-suppressive, antineoplastic agents and combination thereof.

48. The medical implant device according to claim 42 wherein said pharmaceutical composition further comprises a second biologically active agent selected from the group consisting of hormones, cytokines, growth factors, antibodies, immune-suppressive, antineoplastic agents and combination thereof.
